(19) United States
(12) Patent Application Publication
(10) Pub. No.: US 2006/0088512 A1
(43) Pub. Date: Apr. 27, 2006

(54) Treatment of T cell disorders

(75) Inventor: Richard Boyd, Victoria (AU)

Correspondence Address: WILMER CUTLER PICKERING HALE AND DORR LLP, 60 STATE STREET, BOSTON, MA, 02109, US

(73) Assignee: Monash University, Clayton (AU)

(21) Appl. No.: 11/296,676

(22) Filed: Dec. 7, 2005

Related U.S. Application Data
(63) Continuation of application No. 10/399,213, filed on Feb. 13, 2004.

(51) Int. Cl. A61K 48/00; A61K 38/09; A61K 31/473

(52) U.S. Cl. 424/93.21; 424/93.7; 514/15; 514/284

(57) ABSTRACT

The present invention relates to a method for treating a T cell disorder in a subject involving disrupting sex steroid signaling to the thymus and introducing into the subject bone marrow or haemopoietic stem cells (HSC).

### FIELD OF THE INVENTION

The present invention relates to a method for treating a T cell disorder in a subject involving disrupting sex steroid signalling to the thymus and introducing into the subject bone marrow or haemopoietic stem cells (HSC).

### BACKGROUND OF THE INVENTION

The thymus is influenced to a great extent by its bidirectional communication with the neuroendocrine system (Kendall, 1988). Of particular importance is the interplay between the pituitary, adrenals and gonads on thymic function including both trophic (TSH and GH) and atrophic effects (LH, FSH and ACTH) (Kendall, 1988; Homo-Delarche, 1991). Indeed one of the characteristic features of thymic physiology is the progressive decline in structure and function which is commensurate with the increase in circulating sex steroid production around puberty (Hirokawa and Makinodan, 1975; Tosi et al., 1982 and Hirokawa, et al., 1994). The precise target of the hormones and the mechanism by which they induce thymus atrophy is yet to be determined. Since the thymus is the primary site for the production and maintenance of the peripheral T cell pool, this atrophy has been widely postulated as the primary cause of an increased incidence of immune-based disorders in the elderly. In particular, deficiencies of the immune system illustrated by a decrease in T-cell dependent immune functions such as cytolytic T-cell activity and mitogenic responses, are reflected by an increased incidence of immunodeficiency, autoimmunity and tumour load in later life (Hirokawa, 1998).

The impact of thymus atrophy is reflected in the periphery, with reduced thymic input to the T cell pool resulting in a less diverse T cell receptor (TCR) repertoire. Altered cytokine profile (Hobbs et al., 1993; Kurashima et al., 1995); changes in CD4⁺ and CD8⁺ subsets and a bias towards memory as opposed to naive T cells (Mackall et al., 1995) are also observed. Furthermore, the efficiency of thymopoiesis is impaired with age such that the ability of the immune system to regenerate normal T-cell numbers after T-cell depletion, is eventually lost (Mackall et al., 1995). However, recent work by Douek et al. (1998), has shown presumably thymic output to occur even in old age in humans. Excisional DNA products of TCR gene-rearrangement were used to demonstrate circulating, de novo produced naive T cells after HIV infection in older patients. The rate of this output and subsequent peripheral T cell pool regeneration needs to be further addressed since patients who have undergone chemotherapy show a greatly reduced rate of regeneration of the T cell pool, particularly CD4⁺ T cells, in post-pubertal patients compared to those who were pre-pubertal (Mackall et al, 1995). This is further exemplified in recent work by Timm and Thoman (1999), who have shown that although CD4⁺ T cells are regenerated in old mice post BMT, they appear to show a bias towards memory cells due to the aged peripheral microenvironment, coupled to poor thymic production of naive T cells.

The thymus essentially consists of developing thymocytes interspersed within the diverse stromal cells (predominantly epithelial cell subsets) which constitute the microenvironment and provide the growth factors and cellular interactions necessary for the optimal development of the T cells. The symbiotic developmental relationship between thymocytes and the epithelial subsets that controls their differentiation and maturation (Boyd et al., 1993), means sex-steroid inhibition could occur at the level of either cell type which would then influence the status of the other. It is less likely that there is an inherent defect within the thymocytes themselves since previous studies, utilising radiation chimeras, have shown that BM stem cells are not affected by age (Hirokawa, 1998; Mackall and Gress, 1997) and have a similar degree of thymus repopulation potential as young BM cells. Furthermore, thymocytes in older aged animals retain their ability to differentiate to at least some degree (Mackall and Gress, 1997; George and Ritter, 1996; Hirokawa et al., 1994). However, recent work by Aspinall (1997), has shown a defect within the precursor CD3⁻CD4⁻CD8⁻ triple negative (TN) population occurring at the stage of TCR β chain gene-rearrangement

In the particular case for AIDS, the primary defect in the immune system is the destruction of CD4+ cells and to a lesser extent the cells of the myleoid lineages of macrophages and dendritic cells (DC). Without these the immune system is paralysed and the patient is extremely susceptible to opportunistic infection with death a common consequence. The present treatment for AIDS is based on a multitude of anti-viral drugs to kill or deplete the HIV virus. Such therapies are now becoming more effective with viral loads being reduced dramatically to the point where the patient can be deemed as being in remission. The major problem of immune deficiency still exists however, because there are still very few functional T cells, and those which do recover, do so very slowly. The period of immune deficiency is thus still a very long time and in some cases immune defence mechanisms may never recover sufficiently. The reason for this is that in post-pubertal people the thymus is atrophied.

To generate new T lymphocytes, the thymus requires precursor cells; these can be derived from within the organ itself for a short time, but we have shown that by 3-4 weeks, such cells are depleted and new HSC must be taken in (under normal circumstances this would be from the bone marrow via the blood). However, even in a normal functional young thymus, the intake of such cells is very low (sufficient to maintain T cell production at homeostatically regulated levels. Indeed the entry of cells into the thymus is extremely limited and effectively restricted to HSC (or at least prothymocytes which already have a preferential development along the T cell lineage). In the case of the thymus undergoing rejuvenation due a loss of sex steroid inhibition, we have demonstrated that this organ is now very receptive to new precursor cells circulating in the blood, such that the new T cells which develop from both intrathymic and external precursors. By increasing the level of the blood precursor cells, the T cells derived from them will progressively dominate the T cell pool. This means that any gene introduced into the precursors (HSC) will be passed onto all progeny T cells and eventually be present in virtually all of the T cell pool. The level of dominance of these cells over those derived from endogenous host HSC can be easily increased to very high levels by simply increasing the number of transferred exogenous HSC.

### SUMMARY OF THE INVENTION

The present inventors have demonstrated that thymic atrophy (aged induced or as a consequence of conditions such as chemotherapy or radiotherapy) can be profoundly reversed by inhibition of sex steroid production, with virtually complete restoration of thymic structure and function. The present inventors have also found that the basis for this thymus regeneration is in part due to the initial expansion of precursor cells which are derived both intrathymically and via the blood stream. This finding suggests that is possible to seed the thymus with exogenous haemopoietic stem cells (HSC) which have been injected into the subject.

The ability to seed the thymus with genetically modified or exogenous HSC by disrupting sex steroid signalling to the thymus, means that gene therapy in the HSC may be used more efficiently to treat T cell (and myeloid cells which develop in the thymus) disorders. HSC stem cell therapy has met with little or no success to date because the thymus is dormant and incapable of taking up many if any HSC, with T cell production less than 1% of normal levels.

Accordingly, in a first aspect the present invention provides a method of treating a T-cell disorder in a subject, the method comprising disrupting sex steroid signalling to the thymus in the subject and transplanting into the subject bone marrow or HSC.

In a preferred embodiment the T cell disorder is selected from the group consisting of viral infections, such as human immunodeficiency virus infection, a T cell proliferative disease or any disease which reduces T cells numerically or functionally, directly or indirectly. Preferably, the subject has AIDS and has had the viral load reduced by anti-viral treatment.

In a further preferred embodiment, the subject is post-pubertal.

Preferably, inhibition of sex steroid production is achieved by either castration or administration of a sex steroid analogue(s).

Preferred sex steroid analogues include, eulexin, goserelin, leuprolide, dioxalan derivatives such as triptorelin, meterelin, buserelin, histrelin, nafarelin, lutrelin, leuprorelin, and luteinizing hormone-releasing hormone analogues. Currently, it is preferred that sex steroid analogue is an analogue of luteinizing hormone-releasing hormone. More preferably, the luteinizing hormone-releasing hormone analogue is deslorelin.

In yet another preferred embodiment, the sex steroid analogue(s) is administered by a sustained peptide-release formulation. Examples of sustained peptide-release formulations are provided in WO 98/08533, the entire contents of which are incorporated herein by reference.

In a preferred embodiment, the method comprises transplanting enriched HSC into the subject. The HSC may be autologous or heterologous, although it is preferred that the HSC are autologous.

In cases where the subject is infected with HIV, it is preferred that the HSC are genetically modified such that they and their progeny, in particular T cells, macrophages and dendritic cells, are resistant to infection and/or destruction with the HIV virus. The genetic modification may involve introduction into HSC one or more nucleic acid molecules which prevent viral replication, assembly and/or infection. The nucleic acid molecule may be a gene which enclodes an antiviral protein, an antisense construct, a ribozyme, a dsRNA and a catalytic nucleic acid molecule

In cases where the subject has defective T cells, it is preferred that the HSC are genetically modified to normalise the defect. For diseases such as T cell leukaemias, the modification may include the introduction of nucleic acid constructs or genes which normalise the HSC and inhibit or reduce its likelihood of becoming a cancer cell.

It will be appreciated by those skilled in the art that the present method may be useful in treating any T cell cell disorder which has a defined genetic basis. The preferred method involves reactivating thymic function through inhibition of sex steroids to increase the uptake of blood-borne haemopoietic stem cells (HSC). In general, after the onset of puberty, the thymus undergoes severe atrophy under the influence of sex steroids, with its cellular production reduced to less than 1% of the pre-pubertal thymus. The present invention is based on the finding that the inhibition of production of sex steroids releases the thymic inhibition and allows a full regeneration of its function, including increased uptake of blood-derived HSC. The origin of the HSC can be directly from injection or from the bone marrow following prior injection. It is envisaged that blood cells derived from modified HSC will pass the genetic modification onto their progeny cells, including HSC derived from self-renewal, and that the development of these HSC along the T cell and dendritic cell lineages in the thymus is greatly enhanced if not fully facilitated by reactiving thymic function through inhibition of sex steroids.

The method of the present invention is particularly for treatment of AIDS, where the treatment preferably involves reduction of viral load, reactivation of thymic function through inhibition of sex steroids and transfer into the patients of HSC (autologous or from a second party donor) which have been genetically modifed such that all progeny (especially T cells, DC) are resistant to further HIV infection. This means that not only will the patient be depleted of HIV virus and no longer susceptible to general infections because the T cells have returned to normal levels, but the new T cells being resistant to HIV will be able to remove any remnant viral infected cells. In principle a similar strategy could be applied to gene therapy in HSC for any T cell defect or any viral infection which targets T cells.

### BRIEF DESCRIPTION OF THE ACCOMPANYING FIGURES

FIG. 1: Aged (2-year old) mice were surgically castrated and analysed for (a) thymus weight in relation to body weight and (b) total cells per thymus at 2-4 weeks post-castration. A significant decrease in thymus weight and cellularity was seen with age compared to young adult (2-month) mice. This was restored by castration. At 3-weeks post-castration thymic hypertrophy was observed and was returned to young adult levels by 4-weeks post-castration. Results are expressed as mean±1SD of 4-8 mice per group. **=p≦0.01; ***=p≦0.001 compared to young adult and post-castration mice.

FIG. 2: Aged (2-year old) mice were surgically castrated and analysed at 2 and 4 weeks post-castration for peripheral lymphocyte populations. (a) Total lymphocyte numbers in the spleen. No change in total spleen cell numbers was observed with age or post-castration, due to peripheral homeostasis. (b) The ratio of B cells to T cells did not change with age or post-castration, however (c) A significant decrease in the CD4+:CD8+ T cell ratio was seen with age. This was restored by 4-weeks post-castration. Data is expressed as mean±1SD of 4-8 mice per group. ***=p≦0.001 compared to young adult (2-month) and 4-week post-castrate mice.

FIG. 3: Aged (2-year old) mice were castrated and the thymocyte subsets analysed based on the markers CD4 and CD8. Representative FAGS profiles of CD4/CD8 dot plots are shown for CD4−CD8−DN, CD4+ CD8+DP, CD4+ CD8- and CD4−CD8+ SP thymocytes. No difference was seen in the proportions of any CD4/CD8 defined subset with age or post-castration.

FIG. 4.1: Aged (2-year old) mice were castrated and injected with a pulse of bromodeoxyuridine (BrdU) to determine levels of proliferation. Representative histogram profiles of the proportion of BrdU+ cells within the thymus with age and post-castration are shown. No difference in the proportion of proliferating cells within the total thymus was observed with age or post-castration.

FIG. 4.2: Aged (2-year old) mice were castrated and injected with a pulse of bromodeoxyuridine (BrdU) to determine levels of proliferation. Analysis of proliferation within the different subsets of thymocytes based on CD4 and CD8 expression within the thymus was performed. (a) The proportion of each thymocyte subset within the BrdU+ population did not change with age or post-castration. (b) However, a significant decrease in the proportion of DN (CD4−CD8−) thymocytes proliferating was seen with age. Post-castration, this was restored and a significant increase in proliferation within the CD4−CD8+ SP thymocytes was observed. (c) No change in the total proportion of BrdU+ cells within the TN subset was seen with age or post-castration. However (d) a significant decrease in proliferation within the TN1 (CD44+CD25−) and significant increase in proliferation within TN2 (CD44+CD25+) subsets was seen with age. This was restored post-castration. Results are expressed as mean±1SD of 4-8 mice per group. *=p≦0.05; ***=p≦0.001 compared to young adult (2-month) mice.

FIG. 5: Aged (2-year old) mice were castrated and were injected intrathymically with FITC to determine thymic export rates. The number of FITC+ cells in the periphery were calculated 24 hours later. (a) A significant decrease in recent thymic emigrant (RTE) cell numbers was observed with age. Following castration, these values had significantly increased by 2 weeks post-cx. (b) The rate of emigration (export/total thymus cellularity) remained constant with age but was significantly reduced at 2 weeks post-cx. (c) With age, a significant increase in the ratio of CD4+ to GD8+ RTE was seen and this was normalised by 1-week post-cx. Results are expressed as mean±1SD of 4-8 mice per group. **=p≦0.01; ***=p≦0.001 compared to young adult mice. ˆ=p≦0.001 compared to castrated mice.

FIG. 6: Young (3-month old) mice were depleted of lymphocytes using cyclophosphamide. Mice were either sham-castrated or castrated on the same day as cyclophosphamide treatment (a) A significant increase in thymus cell number was observed in castrated mice compared to sham-castrated mice. (b) Castrated mice also showed a significant increase in spleen cell number at 1-week post-cyclophosphamide treatment (c) A significant increase in lymph node cellularity was also observed with castrated mice at 1-week post-treatment Results are expressed as mean±1SD of 4-8 mice per group. ***=p≦0.001 compared to castrated mice.

FIG. 7: Young (3-month old) mice were depleted of lymphocytes using sublethal (625 Rads) irradiation. Mice were either sham-castrated or castrated on the same day as irradiation. Castrated mice showed a significantly faster rate of thymus regeneration compared to sham-castrated counterparts (a). No difference in spleen (b) or lymph node (c) cell numbers was seen with castrated mice. Lymph node cell numbers were still chronically low at 2-weeks post-treatment compared to control mice. Results are expressed as mean±1SD of 4-8 mice per group. *=p≦0.05 compared to control mice; ***=p≦0.001 compared to control and castrated mice.

FIG. 8: Young (3-month old) mice were depleted of lymphocytes using sublethal (625 Rads) irradiation. Mice were either sham-castrated or castrated 1-week prior to irradiation. A significant increase in thymus regeneration was observed with castration (a). No difference in spleen (b) or lymph node (c) cell numbers was seen with castrated mice. Lymph node cell numbers were still chronically low at 2-weeks post-treatment compared to control mice. Results are expressed as mean±1SD of 4-8 mice per group. *=p≦0.05; **=p≦0.01 compared to control mice; ***=p≦0.001 compared to control and castrated mice.

FIG. 9: Changes in thymus, spleen and lymph node cell numbers following treatment with cyclophosphamide, a chemotherapy agent, and surgical or chemical castration performed on the same day. Note the rapid expansion of the thymus in castrated animals when compared to the non-castrate (cyclophosphamide alone) group at 1 and 2 weeks post-treatment. In addition, spleen and lymph node numbers of the castrate group were well increased compared to the cyclophosphamide alone group. (n=3-4 per treatment group and time point). Chemical castration is comparable to surgical castration in regeneration of the immune system post-cyclophosphamide treatment.

FIG. 10: Aged mice (2-years) were castrated and analysed for response to Herpes Simplex Virus-1. (a) Aged mice showed a significant reduction in total lymph node cellularity post-infection when compared to both the young and post-castrate mice. (b) Representative FACS profiles of activated (CD8+ CD25+) cells in the LN of HSV-1 infected mice. No difference was seen in proportions of activated CTL with age or post-castration. (c) The decreased cellularity within the lymph nodes of aged mice was reflected by a significant decrease in activated CTL numbers. Castration of the aged mice restored the immune response to HSV-1 with activated cell numbers equivalent to young mice. Results are expressed as mean±1SD of 8-12 mice. **=p≦0.01 compared to both young (2-month) and castrated mice.

FIG. 11: Popliteal lymph nodes were removed from mice immunised with HSV-1 and cultured for 3-days. CTL assays were performed with non-immunised mice as control for background levels of lysis (as determined by 51Cr-release. Results are expressed as mean of 8 mice, in triplicate +1SD. Aged mice showed a significant reduction in CTL activity at an E:T ratio of both 10:1 and 3:1 indicating a reduction in the percentage of specific CTL present within the lymph nodes. Castration of aged mice restored the CTL response to young adult levels. *=p≦0.01 compared to young adult and post-castrate aged mice.

FIG. 12: Analysis of CD4+ T cell help and VP TCR response to HSV-1 infection. Popliteal lymph nodes were removed on D5 post-HSV-1 infection and analysed ex-vivo for the expression of (a) CD25, CD8 and specific TCRVβmarkers and (b) CD4/CD8 T cells. (a) The percentage of activated (CD25+) CD8+ T cells expressing either Vβ10 or Vβ8.1 is shown as mean±1SD for 8 mice per group. No difference was observed with age or post-castration. (b) A decrease in CD4/CD8 ratio in the resting LN population was seen with age. This was restored post-castration. Results are expressed as mean±1SD of 8 mice per group. ***=p≦0.001 compared to young and castrate mice.

FIG. 13: Vβ10 expression on CTL in activated LN following HSV-1 inoculation. Despite the normal Vβ10 responsiveness in aged mice overall, in some mice a complete loss of Vβ10 expression was observed. Representative histogram profiles are shown. Note the diminution of a clonal response in aged mice and the reinstatement of the expected response post-castration.

FIG. 14: Changes in thymus, spleen, lymph node and bone marrow cell numbers following bone marrow transplantation of Ly5 congenic mice. Note the rapid expansion of the thymus in castrated animals when compared to the non-castrate group at all time points post-treatment In addition, spleen and lymph node numbers of the castrate group were well increased compared to the cyclophosphamide alone group. (n=3-4 per treatment group and time point). Castrated mice had significantly increased congenic (Ly5.2) cells compared to non-castrated animals (data not shown).

FIG. 15: Changes in thymus cell number in castrated and non-castrated mice after fetal liver reconstitution. (n=3-4 for each test group.) (a) At two weeks, thymus cell number of castrated mice was at normal levels and significantly higher than that of non-castrated mice (*p<0.05). Hypertrophy was observed in thymuses of castrated mice after four weeks. Non-castrated cell numbers remain below control levels. (b) CD45.2+ cells −CD45.2+is a marker showing donor derivation. Two weeks after reconstitution donor-derived cells were present in both castrated and non-castrated mice. Four weeks after treatment approximately 85% of cells in the castrated thymus were donor-derived. There were no donor-derived cells in the non-castrated thymus.

FIG. 16: FACS profiles of CD4 versus CD8 donor derived thymocyte populations after lethal irradiation and fetal liver reconstitution, followed by surgical castration. Percentages for each quadrant are given to the right of each plot. The age matched control profile is of an eight-month old Ly5.1 congenic mouse thymus. Those of castrated and non-castrated mice are gated on CD45:2+ cells, showing only donor derived cells. Two weeks after reconstitution subpopulations of thymocytes do not differ between castrated and non-castrated mice.

FIG. 17: Myeloid and lymphoid dendritic cell (DC) number after lethal irradiation, fetal liver reconstitution and castration. (n=3-4 mice for each test group.) Control (white) bars on the following graphs are based on the normal number of dendritic cells found in untreated age matched mice. (a) Donor-derived myeloid dendritic cells—Two weeks after reconstitution DC were present at normal levels in non-castrated mice. There was significantly more DC in castrated mice at the same time point. (*p<0.05). At four weeks DC number remained above control levels in castrated mice. (b) Donor-derived lymphoid dendritic cells—Two weeks after reconstitution, DC numbers in castrated mice were double those of non-castrated mice. Four weeks after treatment DC numbers remained above control levels.

FIG. 18: Changes in total and CD45.2+ bone marrow cell numbers in castrated and non-castrated mice after fetal liver reconstitution. n=34 mice for each test group. (a) Total cell number—Two weeks after reconstitution bone marrow cell numbers had normalised and there was no significant difference in cell number between castrated and non-castrated mice. Four weeks after reconstitution there was a significant difference in cell number between castrated and non-castrated mice (*p<0.05). (B) CD45.2+ cell number. There was no significant difference between castrated and non-castrated mice with respect to CD45.2+ cell number in the bone marrow two weeks after reconstitution. CD45.2+ cell number remained high in castrated mice at four weeks. There were no donor-derived cells in the non-castrated mice at the same time point.

FIG. 19: Changes in T cells and myeloid and lymphoid derived dendritic cells. (DC) in bone marrow of castrated and non-castrated mice after fetal liver reconstitution. (n=3-4 mice for each test group.) Controls (white) bars on the following graphs are based on the normal number of T cells and dendritic cells found in untreated age matched mice. (a) T cell number—Numbers were reduced two and four weeks after reconstitution in both castrated and non-castrated mice. (b) Donor derived myeloid dendritic cells—Two weeks after reconstitution DC cell numbers were normal in both castrated and non-castrated mice. At this time point there was no significant difference between numbers in castrated and non-castrated mice. (c) Donor-derived lymphoid dendritic cells—Numbers were at normal levels two and four weeks after reconstitution. At two weeks there was no significant difference between numbers in castrated and non-castrated mice.

FIG. 20: Change in total and donor (CD45.2+) spleen cell numbers in castrated and non-castrated mice after fetal liver reconstitution. (n=3-4 mice for each test group.) (a) Total cell number—Two weeks after reconstitution cell numbers were decreased and there was no significant difference in cell number between castrated and non-castrated mice. Four weeks after reconstitution cell numbers were approaching normal levels in castrated mice. (b) CD45.2+ cell number—There was no significant difference between castrated and non-castrated mice with respect to CD45.2+ cell number in the spleen, two weeks after reconstitution. CD45.2+ cell number remained high in castrated mice at four weeks. There were no donor-derived cells in the non-castrated mice at the same time point

FIG. 21: Splenic T cells and myeloid and lymphoid derived dendritic cells (DC) after fetal liver reconstitution. (n=3-4 mice for each test group.) Control (white) bars on the following graphs are based on the normal number of T cells and dendritic cells found in untreated age matched mice. (a) T cell number—Numbers were reduced two and four weeks after reconstitution in both castrated and non-castrated mice. (b) Donor derived (CD45.2+) myeloid dendritic cells—two and four weeks after reconstitution DC numbers were normal in both castrated and non-castrated mice. At two weeks there was no significant difference between numbers in castrated and non-castrated mice. (c) Donor-derived (CD45.2+) lymphoid dendritic cells—numbers were at normal levels two and four weeks after reconstitution. At two weeks there was no significant difference between numbers in castrated and non-castrated mice.

FIG. 22: Changes in total and donor (CD45.2+) lymph node cell numbers in castrated and non-castrated mice after fetal liver reconstitution. (n=3-4 for each test group.) (a) Total cell numbers—Two weeks after reconstitution cell numbers were at normal levels and there was no significant difference between castrated and non-castrated mice. Four weeks after reconstitution cell numbers in castrated mice were at normal levels. (b) CD45.2+ cell number—There was no significant difference between castrated and non-castrated mice with respect to donor CD45.2+ cell number in the lymph node two weeks after reconstitution. CD45.2 cell number remained high in castrated mice at four weeks. There were no donor-derived cells in the non-castrated mice at the same point

FIG. 23: Changes in T cells and myeloid and lymphoid derived dendritic cells (DC) in the mesenteric lymph nodes of castrated and non-castrated mice after fetal liver reconstitution. (n=34 mice for each test group.) Control (white) bars are the numbers of T cells and dendritic cells found in untreated age matched mice. (a) T cell numbers were reduced two and four weeks after reconstitution in both castrated and non-castrated mice. (b) Donor derived myeloid dendritic cells were normal in both castrated and non-castrated mice. At four weeks they were decreased. At two weeks there was no significant difference between numbers in castrated and non-castrated mice. (c) Donor-derived lymphoid dendritic cells—Numbers were at normal levels two and four weeks after reconstitution. At two weeks there was no significant difference between numbers in castrated and non-castrated mice.

### DETAILED DESCRIPTION OF THE INVENTION

Definitions

The phrase “modifying the T-cell population makeup” refers to altering the nature and/or ratio of T cell subsets defined functionally and by expression of characteristic molecules. Examples of these characteristic molecules include, but are not limited to, the T cell receptor, CD4, CD8, CD3, CD25, CD28, CD44, CD62L and CD69.

The phrase “increasing the number of T-cells” refers to an absolute increase in the number of T cells in a subject in the thymus and/or in circulation and/or in the spleen and/or in the bone marrow and/or in peripheral tissues such as lymph nodes, gastrointestinal, urogenital and respiratory tracts. This phrase also refers to a relative increase in T cells, for instance when compared to B cells.

A “subject having a depressed or abnormal T-cell population or function” includes an individual infected with the human immunodeficiency virus, especially one who has AIDS, or any other virus or infection which attacks T cells or any T cell disease for which a defective gene has been identified.

Furthermore, this phrase includes any post-pubertal individual, especially an aged person who has decreased immune responsiveness and increased incidence of disease as a consequence of post-pubertal thymic atrophy.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Disruption of Sex Steroid Signalling

As will be readily understood, sex steroid signalling to the thymus can be disrupted in a range of ways, for example, inhibition of sex steroid production or blocking a sex steroid receptor(s) within the thymus. Inhibition of sex steroid production can be achieved, for example, by castration, administration of a sex steroid analogue(s), and other well known techniques. In some clinical cases permanent removal of the gonads via physical castration may be appropriate. In a preferred embodiment, the sex steroid signalling to the thymus is disrupted by administration of a sex steroid analogue, preferably an analogue of luteinizing hormone-releasing hormone. It is currently preferred that the analogue is deslorelin (described in U.S. Pat. No. 4,218,439).

Sex Steroid Analogues

Sex steroid analogues and their use in therapies and “chemical castration” are well known. Examples of such analogues include Eulexin (described in FR7923545, WO 86/01105 and PT100899), Goserelin (described in U.S. Pat. No. 4,100,274, U.S. Pat. No. 4,128,638, GB9112859 and GB9112825), Leuprolide (described in U.S. Pat. No. 4,490,291, U.S. Pat. No. 3,972,859, U.S. Pat. No. 4,008,209, U.S. Pat. No. 4,005,063, DE2509783 and U.S. Pat. No. 4,992,421), dioxalan derivatives such as are described in EP 413209, Triptorelin (described in U.S. Pat. No. 4,010,125, U.S. Pat. No. 4,018,726, U.S. Pat. No. 4,024,121, EP 364819 and U.S. Pat. No. 5,258,492), Meterelin (described in EP 23904), Buserelin (described in U.S. Pat. No. 4,003,884, U.S. Pat. No. 4,118,483 and U.S. Pat. No. 4,275,001), Histrelin (described in EP217659), Nafarelin (described in U.S. Pat. No. 4,234,571, WO93/15722 and EP52510), Lutrelin (described in U.S. Pat. No. 4,089,946), Leuprorelin (described in Plosker et al.) and LHRH analogues such as are described in EP181236, U.S. Pat. No. 4,608,251, U.S. Pat. No. 4,656,247, U.S. Pat. No. 4,642,332, U.S. Pat. No. 4,010,149, U.S. Pat. No. 3,992,365 and U.S. Pat. No. 4,010,149. The disclosures of each the references referred to above are incorporated herein by cross reference.

As will be understood by persons skilled in the art at least some of the means for disrupting sex steroid signalling to the thymus will only be effective as long as the appropriate compound is administered. As a result, an advantage of certain embodiments of the present invention is that once the desired immunological affects of the present invention have been achieved, (2-3 months) the treatment can be stopped and the subjects reproductive system will return to normal.

Genetic Modification of Haemopoietic Stem Cells (HSC)

Methods for isolating and transducing stems cells and progenitor cells would be well known to those skilled in the art. Examples of these types of processes are described, for example, in WO 95/08105, U.S. Pat. No. 5,559,703, U.S. Pat. No. 5,399,493, U.S. Pat. No. 5,061,620, WO 96/33281, WO 96/33282, U.S. Pat. No. 5,681,559 and U.S. Pat. No. 5,199,942.

Antisense Polynucleotides

The term “antisense”, as used herein, refers to polynucleotide sequences which are complementary to a polynucleotide of the present invention. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation in this manner, mutant phenotypes may be generated.

Catalytic Nucleic Acids

The term catalytic nucleic acid refers to a DNA molecule or DNA-containing molecule (also known in the art as a “deoxyribozyme” or “DNAzyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T and U, as well as derivatives thereof. Derivatives of these bases are well known in the art.

Typically, the catalytic nucleic acid contains an antisense sequence for specific recognition of a target nucleic acid, and a nucleic acid cleaving enzymatic activity. The catalytic strand cleaves a specific site in a target nucleic acid. The types of ribozymes that are particularly useful in this invention are the hammerhead ribozyme (Haseloff and Gerlach 1988, Perriman et al., 1992) and the hairpin ribozyme (Shippy et al., 1999).

dsRNA

dsRNA is particularly useful for specifically inhibiting the production of a particular protein. Although not wishing to be limited by theory, Dougherty and Parks (1995) have provided a model for the mechanism by which dsRNA can be used to reduce protein production. This model has recently been modified and expanded by Waterhouse et al. (1998). This technology relies on the presence of dsRNA molecules that contain a sequence that is essentially identical to the mRNA of the gene of interest, in this case an mRNA encoding a polypeptide according to the first aspect of the invention. Conveniently, the dsRNA can be produced in a single open reading frame in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Dougherty and Parks (1995), Waterhouse et al. (1998), WO 99/32619, WO 99/53050, WO 99/49029, and WO 01/34815.

Anti-HV Constructs

Those skilled in the art would be able to develop suitable anti-HIV constructs for use in the present invention. Indeed, a number of anti-HIV antisense constructs and ribozymes have already been developed and are described, for example; in U.S. Pat. No. 5,811,275, U.S. Pat. No. 5,741,706, WO 94/26877, AU 56394/94 and U.S. Pat. No. 5,144,019.

### EXAMPLE 1

### Reversal of Aged-Induced Thymic Atrophy

Materials and Methods

Animals

CBA/CAH and C57B16/J male mice were obtained from Central Animal Services, Monash University and were housed under conventional conditions. Ages ranged from 4-6 weeks to 26 months of age and are indicated where relevant.

Castration

Animals were anaesthetised by intraperitoneal injection of 0.3 ml of 0.3 mg xylazine (Rompun; Bayer Australia Ltd., Botany NSW, Australia) and 1.5 mg ketamine hydrochloride (Ketalar; Parke-Davis, Caringbah, NSW, Australia) in saline. Surgical castration was performed by a scrotal incision, revealing the testes, which were tied with suture and then removed along with surrounding fatty tissue.

Bromodeoxyuridine (BrdU) Incorporation

Mice received two intraperitoneal injections of BrdU (Sigma Chemical Co., St Louis, Mo.) (100 mg/kg body weight in 100 μl of PBS) at a 4 hour interval. Control mice received vehicle alone injections. One hour after the second injection, thymuses were dissected and either a cell suspension made for FACS analysis, or immediately embedded in Tissue Tek (O.C.T. compound, Miles INC, Indiana), snap frozen in liquid nitrogen, and stored at −70° C. until use.

Flow Cytometric Analysis

Mice were killed by CO₂ asphyxiation and thymus, spleen and mesenteric lymph nodes were removed. Organs were pushed gently through a 200 μm sieve in cold PBS/1% FCS/0.02% Azide, centrifuged (650 g, 5 min, 4° C.), and resuspended in either PBS/FCS/Az. Spleen cells were incubated in red cell lysis buffer (8.9 g/litre ammonium chloride) for 10 min at 4° C., washed and resuspended in PBS/FCS/Az. Cell concentration and viability were determined in duplicate using a haemocytometer and ethidium bromide/acridine orange and viewed under a fluorescence microscope (Axioskop; Carl Zeiss, Oberkochen, Germany).

For 3-colour immunofluorescence thymocytes were routinely labelled with anti-αβ TCR-FITC or anti-γδ TCR-FITC, anti-CD4-PE and anti-CD8-APC (all obtained from Pharmingen, San Diego, Calif.) followed by flow cytometry analysis. Spleen and lymph node suspensions were labelled with either αβTCR-FITC/CD4-PE/CD8-APC or B220-B (Sigma) with CD4-PE and CD8-APC. B220-B was revealed with streptavidin-Tri-color conjugate purchased from Caltag Laboratories, Inc., Burlingame, Calif.

For BrdU detection, cells were surface labelled with CD4-PE and CD8-APC, followed by fixation and permeabilisation as previously described (Carayon and Bord, 1989). Briefly, stained cells were fixed O/N at 4° C. in 1% PFA/0.01% Tween-20. Washed cells were incubated in 500 μl DNase (100 Kunitz units, Boehringer Mannheim, W. Germany) for 30 mins at 37° C. in order to denature the DNA. Finally, cells were incubated with anti-BrdU-FITC (Becton-Dickinson).

For 4-colour Immunofluorescence thymocytes were labelled for CD3, CD4, CD8, B220 and Mac-1, collectively detected by anti-rat Ig-Cy5 (Amersham, U.K.), and the negative cells (TN) gated for analysis. They were further stained for CD25-PE (Pharmingen) and CD44-B (Pharmingen) followed by Streptavidin-Tri-colour (Caltag, Calif.) as previously described (Godfrey and Zlotnik, 1993). BrdU detection was then performed as described above.

Samples were analysed on a FacsCalibur (Becton-Dickinson). Viable lymphocytes were gated according to 0° and 90° light scatter profiles and data was analysed using Cell quest software (Becton-Dickinson).

Immunohistology

Frozen thymus sections (4 μm) were cut using a cryostat (Leica) and immediately fixed in 100% acetone.

For two-colour immunofluorescence, sections were double-labelled with a panel of monoclonal antibodies: MTS6, 10, 12, 15, 16, 20, 24, 32, 33, 35 and 44 (Godfrey et al., 1990; Table 1) produced in this laboratory and the co-expression of epithelial cell determinants was assessed with a polyvalent rabbit anti-cytokeratin Ab (Dako, Carpinteria, Calif.). Bound mAb was revealed with FITC-conjugated sheep anti-rat Ig (Silenus Laboratories) and anti-cytokeratin was revealed with TRITC-conjugated goat anti-rabbit Ig (Silenus Laboratories).

For bromodeoxyuridine detection sections were stained with either anti-cytokeratin followed by anti-rabbit-TRITC or a specific mAb, which was then revealed with anti-rat Ig-Cy3 (Amersham). BrdU detection was then performed as previously described (Penit et al., 1996). Briefly, sections were fixed in 70% Ethanol for 30 mins. Semi-dried sections were incubated in 4M HCl, neutralised by washing in Borate Buffer (Sigma), followed by two washes in PBS. BrdU was detected using anti-BrdU-FITC (Becton-Dickinson).

For three-colour immunofluorescence, sections were labelled for a specific MTS mAb together with anti-cytokeratin. BrdU detection was then performed as described above.

Sections were analysed using a Leica fluorescent and Nikon confocal microscopes.

Migration Studies

Animals were anaesthetised by intraperitoneal injection of 0.3 ml of 0.3 mg xylazine (Rompun; Bayer Australia Ltd., Botany NSW, Australia) and 1.5 mg ketamine hydrochloride (Ketalar; Parke-Davis, Caringbah, NSW, Australia) in saline.

Details of the FITC labelling of thymocytes technique are similar to those described elsewhere (Scollay et al., 1980; Berzins et al., 1998). Briefly, thymic lobes were exposed and each lobe was injected with approximately 10 μm of 350 μg/ml FITC (in PBS). The wound was closed with a surgical staple, and the mouse was warmed until fully recovered from anaesthesia. Mice were killed by CO₂ asphyxiation approximately 24 h after injection and lymphoid organs were removed for analysis.

After cell counts, samples were stained with anti-CD4-PE and anti-CD8-APC, then analysed by flow cytometry. Migrant cells were identified as live-gated FITC⁺ cells expressing either CD4 or CD8 (to omit autofluorescing cells and doublets). The percentages of FITC⁺ CD4 and CD8 cells were added to provide the total migrant percentage for lymph nodes and spleen, respectively. Calculation of daily export rates was performed as described by Berzins et al. (1998).

Data was analysed using the unpaired student ‘t’ test or nonparametrical Mann-Whitney test was used to determine the statistical significance between control and test results for experiments performed at least in triplicate. Experimental values significantly differing from control values are indicated as follows: *p≦0.05, **p≦0.01 and ***p≦0.001.

Results

The Effect of Age on Thymocyte Populations.

(i) Thymic Weight and Thymocyte Number

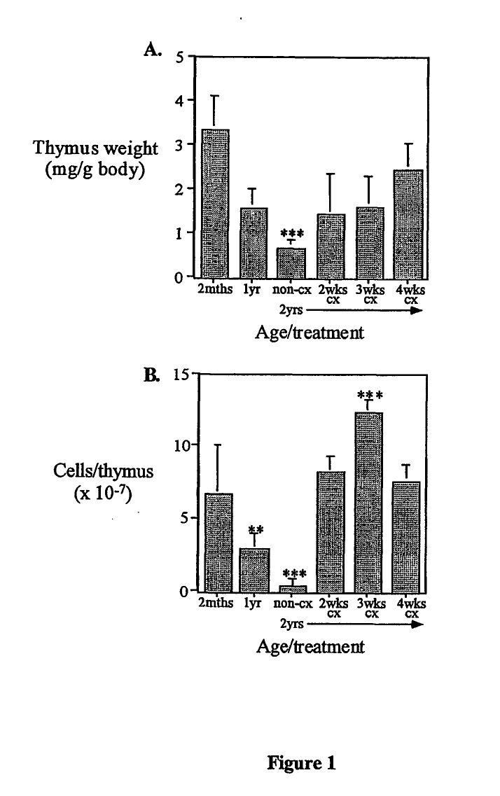

With increasing age there is a highly significant (p≦0.0001) decrease in both thymic weight (FIG. 1A) and total thymocyte number (FIG. 1B). Relative thymic weight (mg thymus/g body) in the young adult has a mean value of 3.34 which decreases to 0.66 at 18-24 months of age (adipose deposition limits accurate calculation). The decrease in thymic weight can be attributed to a decrease in total thymocyte numbers: the 1-2 month thymus contains ˜6.7×10⁷ thymocytes, decreasing to ˜4.5×10⁸ cells by 24 months. By removing the effects of sex steroids on the thymus by castration, regeneration occurs and by 4 weeks post-castration, the thymus is equivalent to that of the young adult in both weight and cellularity (FIGS. 1A and 1B). Interestingly, there is a significant (p≦0.001) increase in thymocyte numbers at 2 weeks post-castration (˜1.2×10⁸), which is restored to normal young levels by 4 weeks post-castration (FIG. 1B).

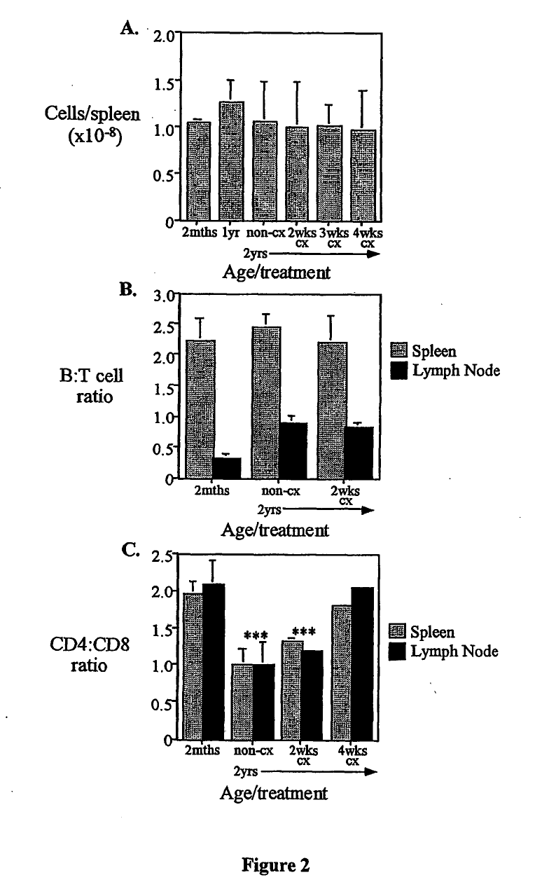

The decrease in T cell numbers produced by the thymus is not reflected in the periphery, with spleen cell numbers remaining constant with age (FIG. 2A). Homeostatic mechanisms in the periphery were evident since the B cell to T cell ratio in spleen and lymph nodes was not affected with age and the subsequent decrease in T cell numbers reaching the periphery (FIG. 2B). However, the ratio of CD4⁺ to CD8⁺ T cell significantly decreased (p≦0.001) with age from 2:1 at 2 months of age, to a ratio of 1:1 at 2 years of age (FIG. 2C). Following castration and the subsequent rise in T cell numbers reaching the periphery, no change in peripheral T cell numbers was observed: splenic T cell numbers and the ratio of B:T cells in both spleen and lymph nodes was not altered following castration (FIGS. 2A and B). The decreased CD4:CD8 ratio in the periphery with age was still evident at 2 weeks post-castration but was completely reversed by 4 weeks post-castration (FIG. 2C).

(ii) αβTCR, γδCR, CD4 and CD8 expression

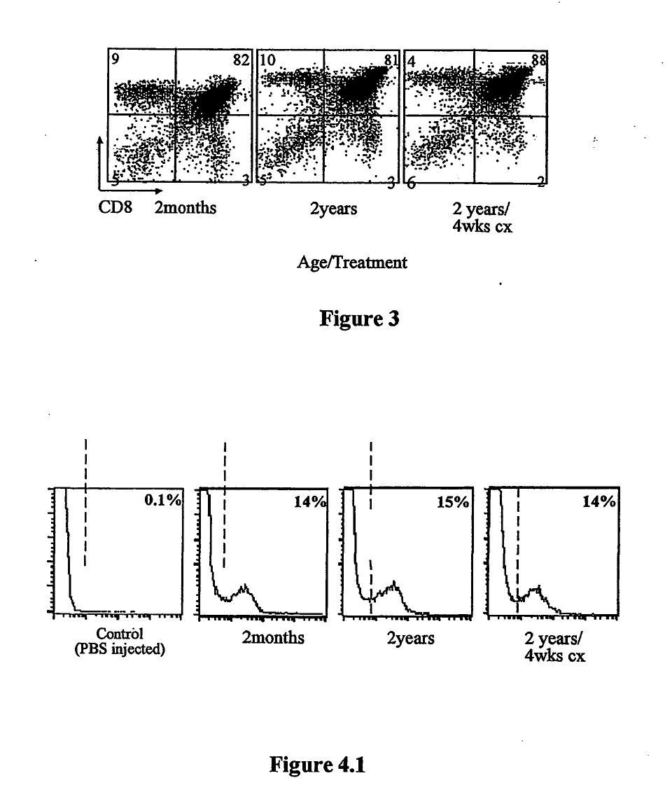

To determine if the decrease in thymocyte numbers seen with age was the result of the depletion of specific cell populations, thymocytes were labelled with defining markers in order to analyse the separate subpopulations. In addition, this allowed analysis of the kinetics of thymus repopulation post-castration. The proportion of the main thymocyte subpopulations was compared with those of the normal young thymus (FIG. 3) and found to remain uniform with age. In addition, further subdivision of thymocytes by the expression of αβTCR and γδTCR revealed no change in the proportions of these populations with age (data not shown). At 2 and 4 weeks post-castration, thymocyte subpopulations remained in the same proportions and, since thymocyte numbers increase by up to 100-fold post-castration, this indicates a synchronous expansion of all thymocyte subsets rather than a developmental progression of expansion.

The decrease in cell numbers seen in the thymus of aged animals thus appears to be the result of a balanced reduction in all cell phenotypes, with no significant changes in T cell populations being detected. Thymus regeneration occurs in a synchronous fashion, replenishing all T cell subpopulations simultaneously rather than sequentially.

Proliferation of Thymocytes

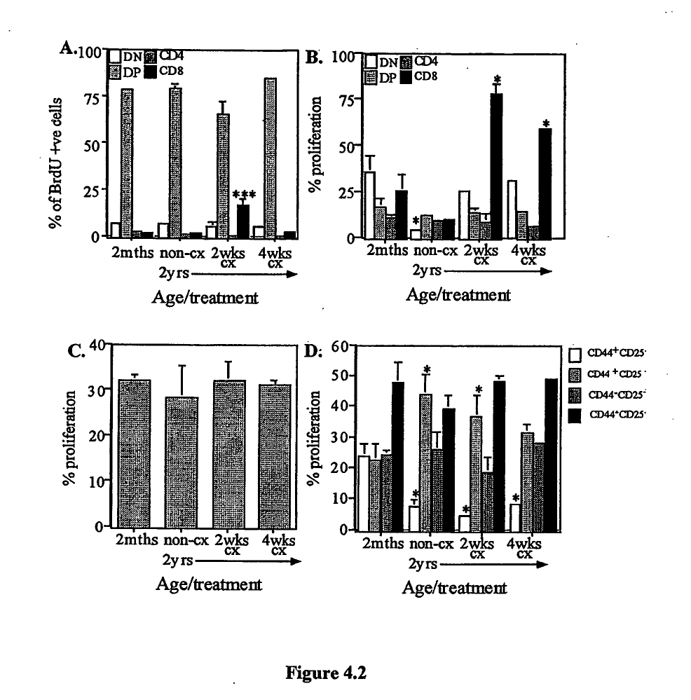

As shown in FIG. 4.1, 15-20% of thymocytes are proliferating at 4-6 weeks of age. The majority (˜80%) of these are DP with the TN subset making up the second largest population at ˜6% (FIG. 4.2A). Accordingly, most division is seen in the subcapsule and cortex by immunohistology (data not shown). Some division is seen in the medullary regions with FACS analysis revealing a proportion of SP cells (9% of CD4 T cells and 25% of CD8 T cells) dividing (FIG. 4.2B).

Although cell numbers are significantly decreased in the aged thymus, proliferation of thymocytes remains constant, decreasing to 12-15% at 2 years (FIG. 4.1), with the phenotype of the proliferating population resembling the 2 month thymus (FIG. 4.2A). Immunohistology revealed the division at 1 year of age to reflect that seen in the young adult, however, at 2 years, proliferation is mainly seen in the outer cortex and surrounding the vasculature (data not shown). At 2 weeks post-castration, although thymocyte numbers significantly increase, there is no change in the proportion of thymocytes that are proliferating, again indicating a synchronous expansion of cells (FIG. 4.1). Immunohistology revealed the localisation of thymocyte proliferation and the extent of dividing cells to resemble the situation in the 2-month-old thymus by 2 weeks post-castration (data not shown). When analysing the proportion of each subpopulation which represent the proliferating population, there was a significant (p<0.001) increase in the percentage of CD8 T cells which are within the proliferating population (1% at 2 months and 2 years of age, increasing to ˜6% at 2 weeks post-castration) (FIG. 4.2A).

FIG. 4.2B illustrates the extent of proliferation within each subset in young, old and castrated mice. There is a significant (p≧0.001) decay in proliferation within the DN subset (35% at 2 months to 4% by 2 years). Proliferation of CD8⁺ T cells was also significantly (p≦0.001) decreased, reflecting the findings by immunohistology (data not shown) where no division is evident in the medulla of the aged thymus. The decrease in DN proliferation is not returned to normal young levels by 4 weeks post-castration. However, proliferation within the CD8⁺ T cell subset is significantly (p≦0.001) increased at 2 weeks post-castration and is returning to normal young levels at 4 weeks post-castration.

The decrease in proliferation within the DN subset was analysed further using the markers CD44 and CD25. The DN subpopulation, in addition to the thymocyte precursors, contains αβTCR⁺CD4⁻CD8⁻ thymocytes, which are thought to have downregulated both co-receptors at the transition to SP cells (Godfrey & Zlotnik, 1993). By gating on these mature cells, it was possible to analyse the true TN compartment (CD3⁻CD4⁻CD8⁻) and these showed no difference in their proliferation rates with age or following castration (FIG. 4.2C). However, analysis of the subpopulations expressing CD44 and CD25, showed a significant (p≦0.001) decrease in proliferation of the TN1 subset (CD44⁺CD25⁻), from 20% in the normal young to around 6% at 18 months of age (FIG. 4.2D) which was restored by 4 weeks post-castration. The decrease in the proliferation of the TN1 subset, was compensated for by a significant (p≦0.001) increase in proliferation of the TN2 subpopulation (CD44⁺CD25⁺) which returned to normal young levels by 2 weeks post-castration (FIG. 4.2D).

The Effect of Age on the Thymic Microenvironment

The changes in the thymic microenvironment with age were examined by immunofluorescence using an extensive panel of mAbs from the MTS series, double-labelled with a polyclonal anti-cytokeratin Ab.

The antigens recognised by these mAbs can be subdivided into three groups: thymic epithelial subsets, vascular-associated antigens and those present on both stromal cells and thymocytes.

(i) Epithelial Cell Antigens.

Anti-keratin staining (pan-epithelium) of 2 year old mouse thymus, revealed a loss of general thymus architecture with a severe epithelial cell disorganisation and absence of a distinct cortico-medullary junction. Further analysis using the mAbs, MTS 10 (medulla) and MTS44 (cortex), showed a distinct reduction in cortex size with age, with a less substantial decrease in medullary epithelium (data not shown). Epithelial cell free regions, or keratin negative areas (KNA's, van Ewijk et al., 1980; Godfrey et al., 1990; Bruijntjes et al., 1993).) were more apparent and increased in size in the aged thymus, as evident with anti-cytokeratin labelling. There is also the appearance of thymic epithelial “cyst-like” structures in the aged thymus particularly noticeable in medullary regions (data not shown). Adipose deposition, severe decrease in thymic size and the decline in integrity of the cortico-medullary junction are shown conclusively with the anti-cytokeratin staining (data not shown). The thymus is beginning to regenerate by 2 weeks post-castration. This is evident in the size of the thymic lobes (a), the increase in cortical epithelium as revealed by MTS 44 (b) and the localisation of medullary epithelium (c). The medullary epithelium is detected by MTS 10 and at 2 weeks, there are still subpockets of epithelium stained by MTS 10 scattered throughout the cortex. By 4 weeks post-castration, there is a distinct medulla and cortex and discernible cortico-medullary junction.

The markers MTS 20 and 24 are presumed to detect primordial epithelial cells (Godfrey, et al.; 1990) and further illustrate the degeneration of the aged thymus. These are present in abundance at E14, detect isolated medullary epithelial cell clusters at 4-6 weeks but are again increased in intensity in the aged thymus (data not shown). Following castration, all these antigens are expressed at a level equivalent to that of the young adult thymus (data not shown) with MTS 20 and MTS 24 reverting to discrete subpockets of epithelium located at the cortico-medullary junction.

(ii) Vascular-Associated Antigens.

The blood-thymus barrier is thought to be responsible for the immigration of T cell precursors to the thymus and the emigration of mature T cells from the thymus to the periphery.

The mAb MTS 15 is specific for the endothelium of thymic blood vessels, demonstrating a granular, diffuse staining pattern (Godfrey, et al., 1990). In the aged thymus, MTS 15 expression is greatly increased, and reflects the increased frequency and size of blood vessels and perivascular spaces (data not shown).

The thymic extracellular matrix, containing important structural and cellular adhesion molecules such as collagen, laminin and fibrinogen, is detected by the mAb MTS 16. Scattered throughout the normal young thymus, the nature of MTS 16 expression becomes more widespread and interconnected in the aged thymus. Expression of MTS 16 is increased further at 2 weeks post-castration while 4 weeks post-castration, this expression is representative of the situation in the 2 month thymus (data not shown).

(iii) Shared Antigens

MHC II expression in the normal young thymus, detected by the mAb MTS6, is strongly positive (granular) on the cortical epithelium (Godfrey et al., 1990) with weaker staining of the medullary epithelium. The aged thymus shows a decrease in MHCII expression with expression substantially increased at 2 weeks post-castration. By 4 weeks post-castration, expression is again reduced and appears similar to the 2 month old thymus (data not shown).

Thymocyte Emigration

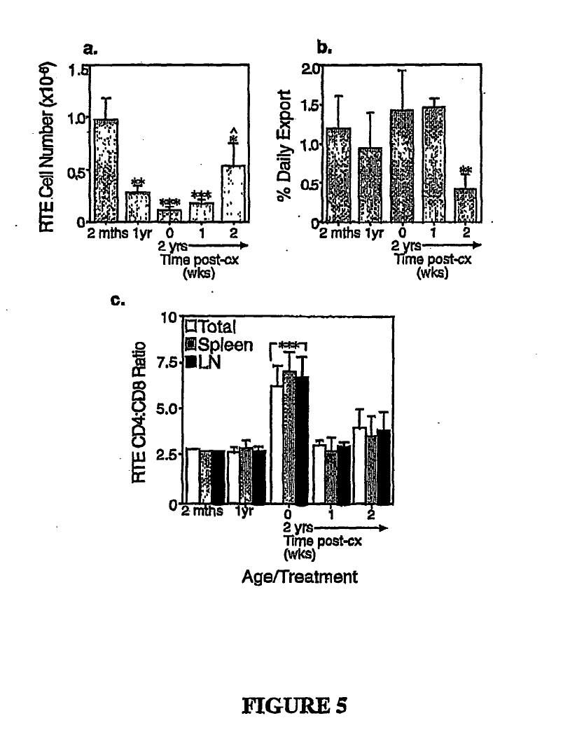

Approximately 1% of T cells emigrate from the thymus daily in the young mouse (Scollay et al., 1980). We found emigration was occurring at a proportional rate equivalent to the normal young mouse at 14 months and even 2 years of age (FIGS. 5a and 5b)) although significantly (p≧0.0001) reduced in number. By 2-weeks post-castration, a significant increase in RTE was observed (p≦0.0001) compared to the aged mice. Despite the changes in cell numbers emigrating, the rate of emigration (RTE/total thymocytes) remained constant with age (FIG. 5b). However, at 2-weeks post-castration this had significantly decreased (p≦0.05), reflecting the increase in total thymocyte nubmers ast this time. Interestingly, there was an increase in the CD4:CD8 ratio of the RTE from ˜3:1 at 2 months to ˜7:1 at 26 months (FIG. 5c). By 1 week post-castration, this ratio had normalised (FIG. 5c).

### EXAMPLE 2

### Reversal of Chemotherapy—or Radiation—Induced Thymic Atrophy

Castrated mice (either one-week prior to treatment, or on the same day as treatment), showed substantial increases in thymus regeneration rate following irradiation or cyclophosphamide treatment

In the thymus, irradiated mice show severe disruption of thymic architecture, concurrent with depletion of rapidly dividing cells. Cortical collapse, reminiscent of the aged/hydrocortisone treated thymus, reveals loss of DN and DP thymocytes. There is a downregulation of αβ-TCR expression on CD4+ and CD8+ SP thymocytes—evidence of apoptosing cells. In comparison, cyclophosphamide-treated animals show a less severe disruption of thymic architecture, and show a faster regeneration rate of DN and DP thymocytes.

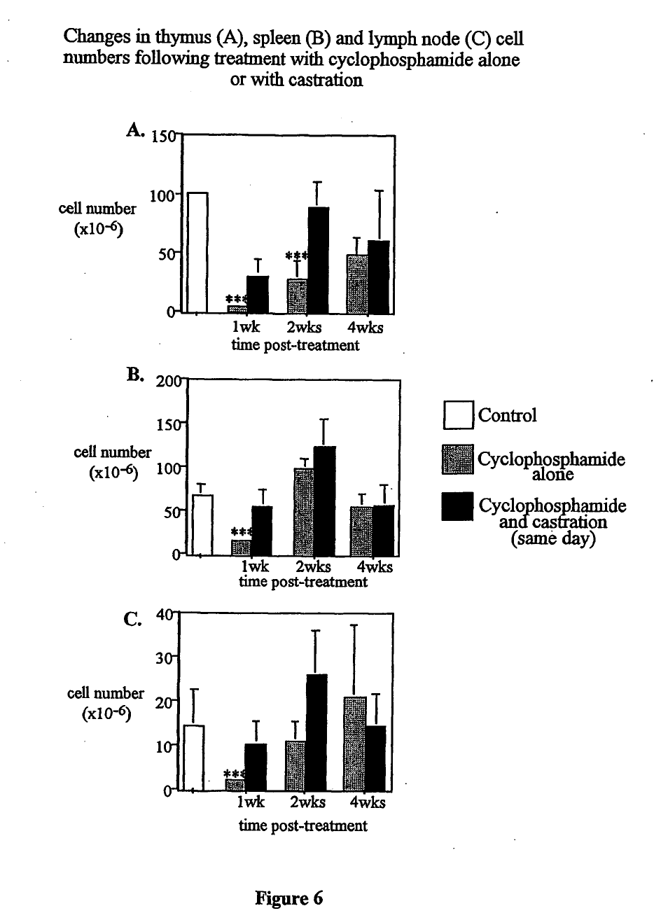

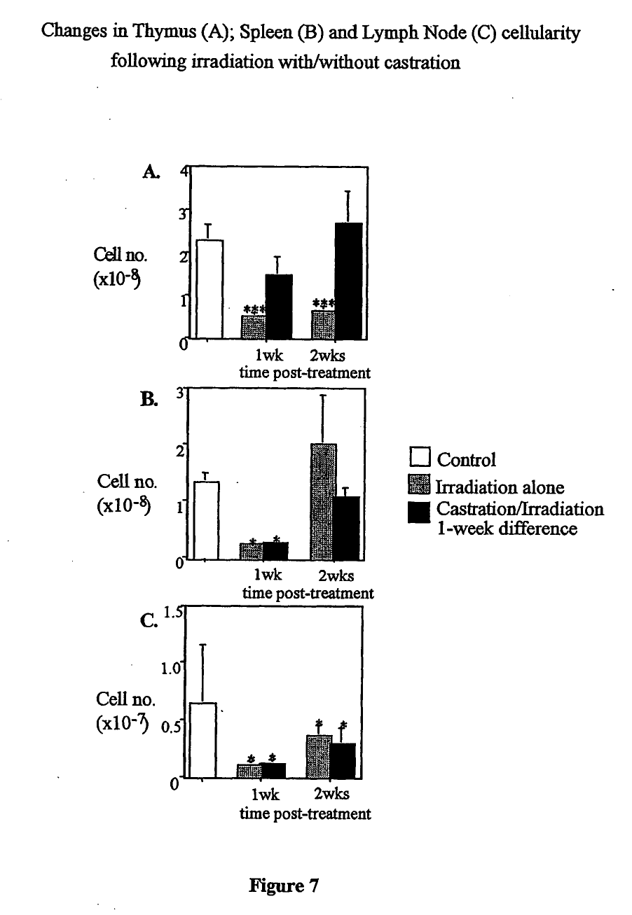

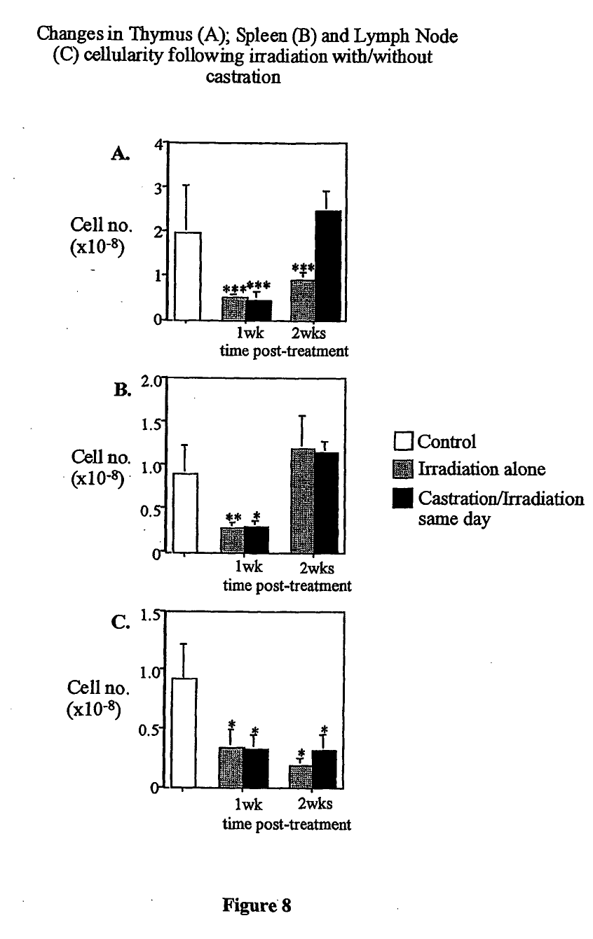

By 1 week post-treatment castrated mice showed significant thymic regeneration even at this early stage (FIGS. 6, 7 and 8). In comparison, non-castrated animals, showed severe loss of DN and DP thymocytes (rapidly-dividing cells) and subsequent increase in proportion of CD4 and CD8 cells (radio-resistant). This is best illustrated by the differences in thymocyte numbers with castrated animals showing at least a 4-fold increase in thymus size even at 1 week post-treatment By 2 weeks, the non-castrated animals showed relative thymocyte normality with regeneration of both DN and DP thymocytes. However, proportions of thymocytes are not yet equivalent to the young adult control thymus. Indeed, at 2 weeks, the vast difference in regulation rates between castrated and non-castrated mice was maximal (by 4 weeks thymocyte numbers were equivalent between treatment groups).

Interestingly, thymus size appears to ‘overshoot’ the baseline of the control thymus. Indicative of rapid expansion within the thymus, with the migration of these newly derived thymocytes not yet occurring (it takes ˜3-4 weeks for thymocytes to migrate through and out into the periphery). Therefore, although proportions within each subpopulation are equal, numbers of thymocytes are building before being released into the periphery.

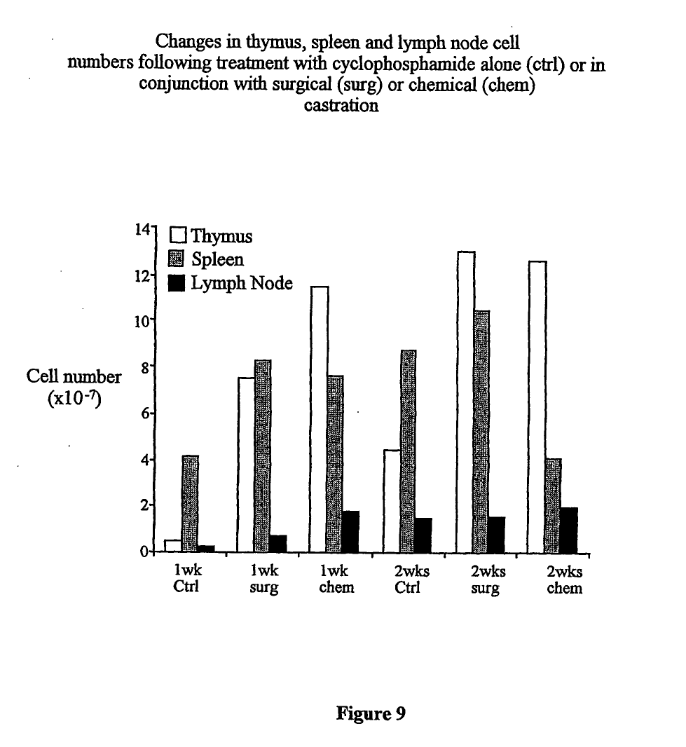

FIG. 9 illustrates the use of chemical castration compared to surgical castration in enhancement of T cell regeneration. The kinetics of chemical castration are much slower than surgical, that is, mice take about 3 weeks longer to decrease their circulating sex steroid levels. However, chemical castration is still effective in regenerating the thymus as illustrated in FIG. 9.

### EXAMPLE 3

### Thymic Regeneration Following Inhibition of Sex Steroids Results in Restoration of Deficient Peripheral T Cell Function

To determine whether castration can enhance the immune response, Herpes Simplex Virus (HSV) immunisation was examined as it allows the study of disease progression and role of CTL (cytotoxic) T cells. Castrated mice have a qualitatively and quantitatively improved responsiveness to the virus. Mice were immunised in the footpad and the popliteal (draining) lymph node analysed at D5 post-immunisation. In addition, the footpad is removed and homogenised to determine the virus titre at particular time-points throughout the experiment.

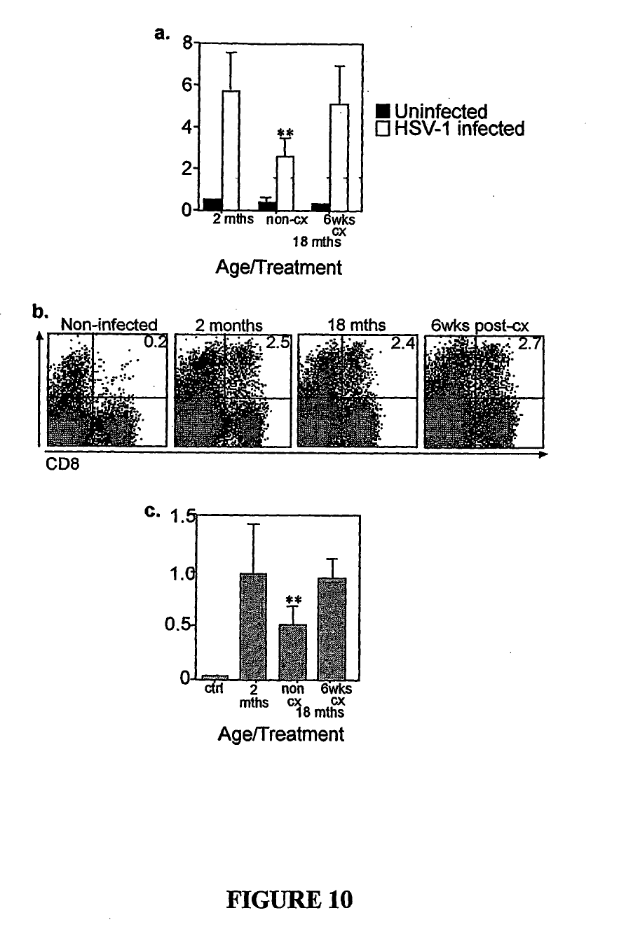

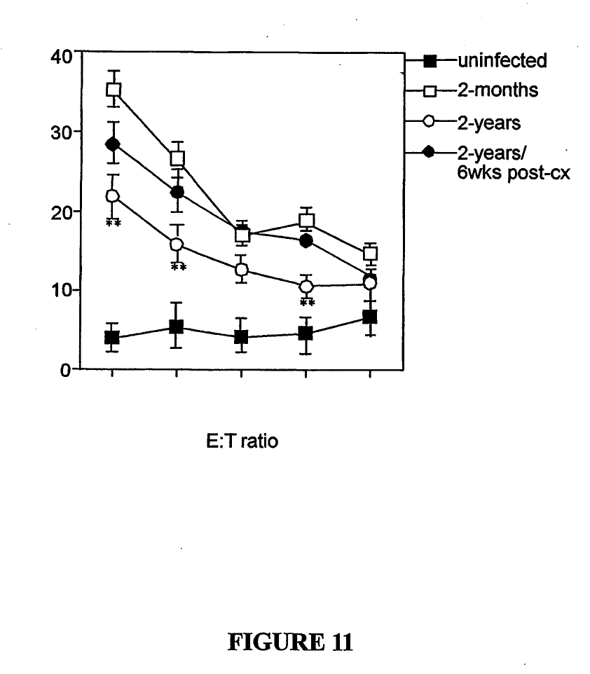

At D5 post-immunisation, the castrated mice have a significantly larger lymph node cellularity than the aged mice (FIG. 10a). Although no difference in the proportion of activated (CD8⁺ CD25⁺) cells was seen with age or post-castration, activated cell numbers within the lymph nodes are significantly increased with castration when compared to the aged controls (FIG. 10c). Further, activated cell numbers correlate with that found for the young adult indicating that CTLs are being activated to a greater extent in the castrated mice, but the young adult may have an enlarged lymph node due to B cell activation. This was confirmed with a CTL assay detecting the proportion of specific lysis occuring with age and post-castration (FIG. 11). Aged mice showed a significantly reduced target cell lysis at effector:target ratios of 10:1 and 3:1 compared to young adult (2-month) mice (FIG. 11). Castration restored the ability of mice to generate specific CTL responses post-HSV infection (FIG. 11).

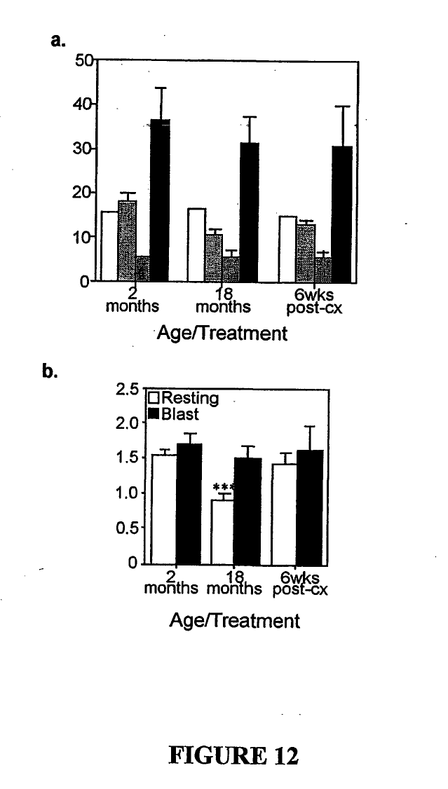

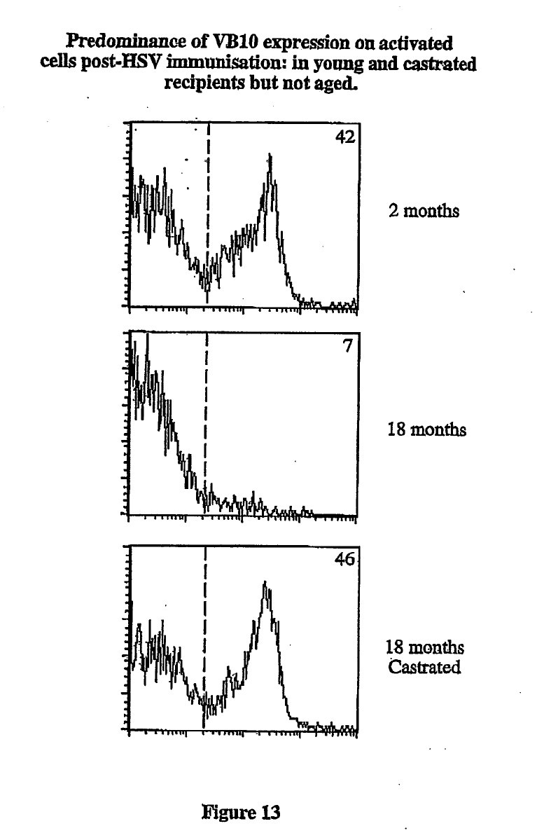

There is a 40% bias post-immunisation for Vβ10 usage for the CTLs in response to HSV. When aged and castrated mice were analysed for their Vβ expression, it was found that this was predominant (FIG. 12a). However, in a sample of aged mice, no such bias was observed (FIG. 13). Furthermore, a decrease in CD4+ T cells in the draining lymph nodes was seen with age compared to both young adult and castrated mice (FIG. 12b). This illustrates the vital need for increased production of T cells from the thymus throughout life, in order to get maximal immune responsiveness.

### EXAMPLE 4

### Inhibition of Sex Steroids Enhances Uptake of New Haemopoietic Precursor Cells Into the Thimus Which Enables Chimeric Mixtures of Host and Donor Lymphoid Cells (T, B, and Dendritic Cells)

Previous experiments have shown that microchimera formation plays an important role in organ transplant acceptance. Dendritic cells have also been shown to play an integral role in tolerance to graft antigens. Therefore, the effects of castration on thymic chimera formation and dendritic cell number was studied.

For the syngeneic experiments, 4 three month old mice were used per treatment group. All controls were age matched and untreated. For congenic experiments, 3-4 eight month old mice were used per treatment group. All controls were age matched and untreated.

Thymic Changes Following Lethal Irradiation, Foetal Liver Reconstitution and Castration of Syngeneic Mice

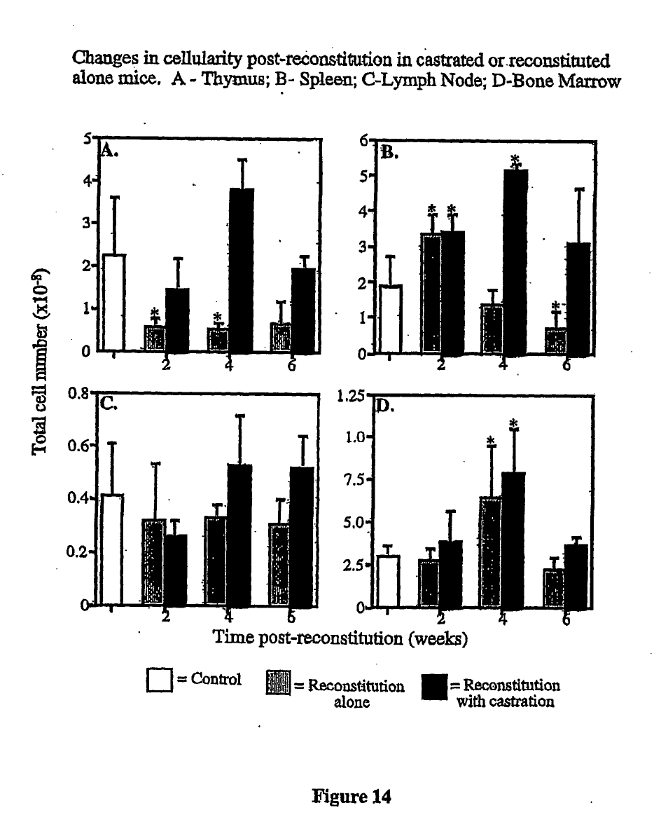

The total thymus cell numbers of castrated and noncastrated reconstituted mice were compared to untreated age matched controls and are summarised in FIG. 14. At both 2- and 4-weeks post-treatment total lymphocyte numbers were significantly increased in castrated compared to noncastrated mice (p≦0.05). At 6 weeks, cell number remained below control levels, however, those of castrated mice was three fold higher than the noncastrated mice (p≦0.05) (FIG. 14A).

Splenic Changes Following Lethal Irradiation, Syngeneic Foetal Liver Reconstitution and Castration.

Total cell numbers in the spleen were greatly decreased 4 and 6 weeks after irradiation and reconstitution, in both castrated and noncastrated mice. Again, castrated mice showed increased lymphocyte numbers at these time-points copared to non-castrated mice (p≦0.05). although no difference in total spleen cell number between castrated and noncastrated treatment groups was seen at 2 weeks (FIG. 14B).

Mesenteric Lymph Nodes Following Lethal Irradiation, Syngeneic Foetal Liver Reconstitution and Castration

Mesenteric lymph node cell numbers were decreased 2-weeks after irradiation and reconstitution, in both castrated and noncastrated mice. However, by the 4 week time point cell numbers had reached control levels. There was no statistically significant difference in lymph node cell number between castrated and noncastrated treatment groups (FIG. 14C).

Thymic Changes Following Lethal Irradiation, Foetal Liver Reconstitution and Castration of Congenic Mice

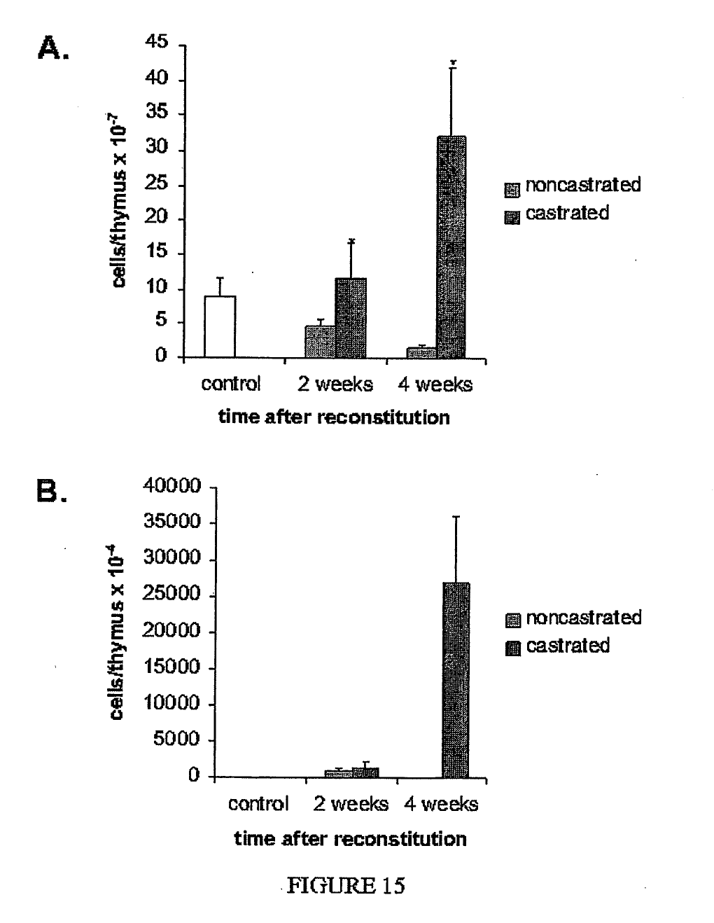

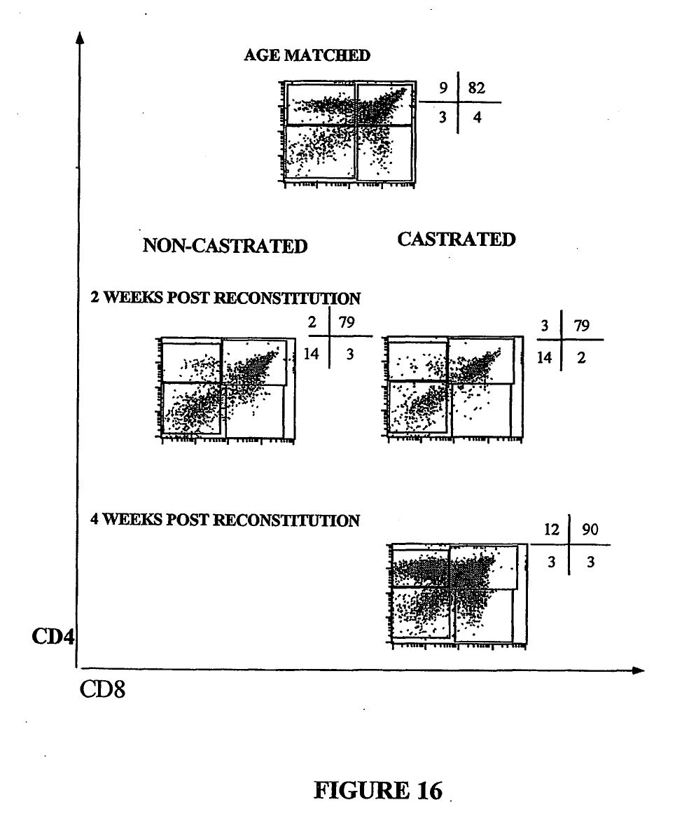

In noncastrated mice, there was a profound decrease in thymocyte number over the 4 week time period, with little or no evidence of regeneration (FIG. 15A). In the castrated group, however, by two weeks there was already extensive thymopoiesis which by four weeks had returned to control levels, being 10 fold higher than in noncastrated mice. Flow cytometeric analysis of the thymii with respect to CD45.2 (donor-derived antigen) demonstrated that no donor derived cells were detectable in the noncastrated group at 4 weeks, but remarkably, virtually all the thymocytes in the castrated mice were donor—derived at this time point (FIG. 15B). Given this extensive enhancement of thymopoiesis from donor-derived haemopoietic precursors, it was important to determine whether T cell differentiation had proceeded normally. CD4, CD8 and TCR defined subsets were analysed by flow cytometry. There were no proportional differences in thymocytes subset proportions 2 weeks after reconstitution (FIG. 16). This observation was not possible at 4 weeks, because the noncastrated mice were not reconstituted with donor derived cells. However, at this time point the thymocyte proportions in castrated mice appear normal.

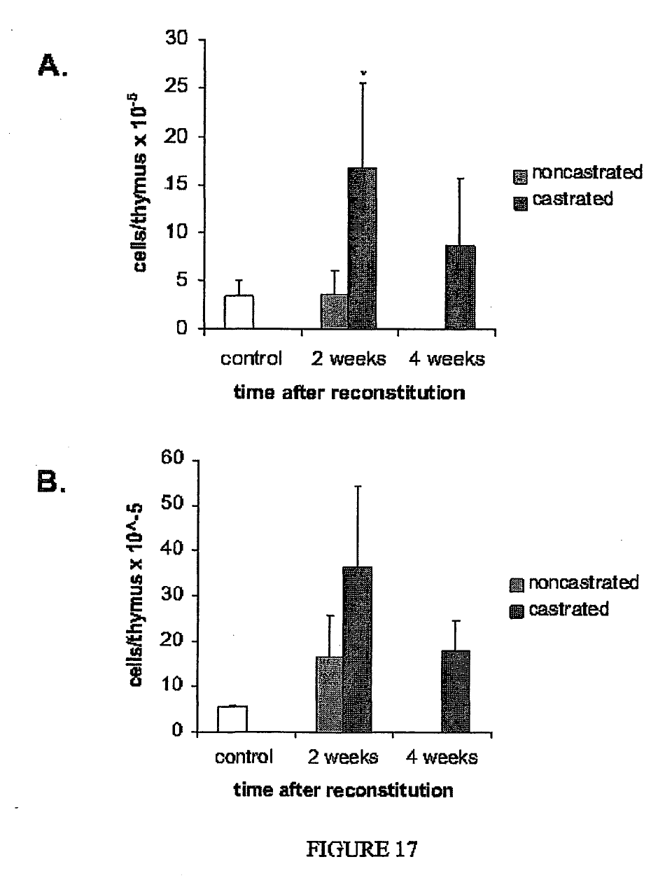

Two weeks after foetal liver reconstitution there were significantly more donor-derived, myeloid dendritic cells (defined as CD45.2+ Mac1+ CD11C+) in castrated mice than noncastrated mice, the difference was 4-fold (p<0.05). Four weeks after treatment the number of donor-derived myeloid dendritic cells remained above the control in castrated mice (FIG. 17A). 2 weeks after foetal liver reconstitution the number of donor derived lymphoid dendritic cells (defined as CD45.2+Mac1−CD11C+) in the thymus of castrated mice was double that found in noncastrated mice. Four weeks after treatment the number of donor-derived lymphoid dendritic cells remained above the control in castrated mice (FIG. 17B).

Immunofluorescent staining for CD11C, epithelium (antikeratin) and CD45.2 (donor-derived marker) localised dendritic cells to the corticomedullary junction and medullary areas of thymii 4 weeks after reconstitution and castration. Using colocalisation software donor-derivation of these cells was confirmed (data not shown). This was supported by flow cytometry data suggesting that 4 weeks after reconstitution approximately 85% of cells in the thymus are donor derived.

Changes in the Bone Marrow Following Lethal Irradiation, Foetal Liver Reconstitution and Castration

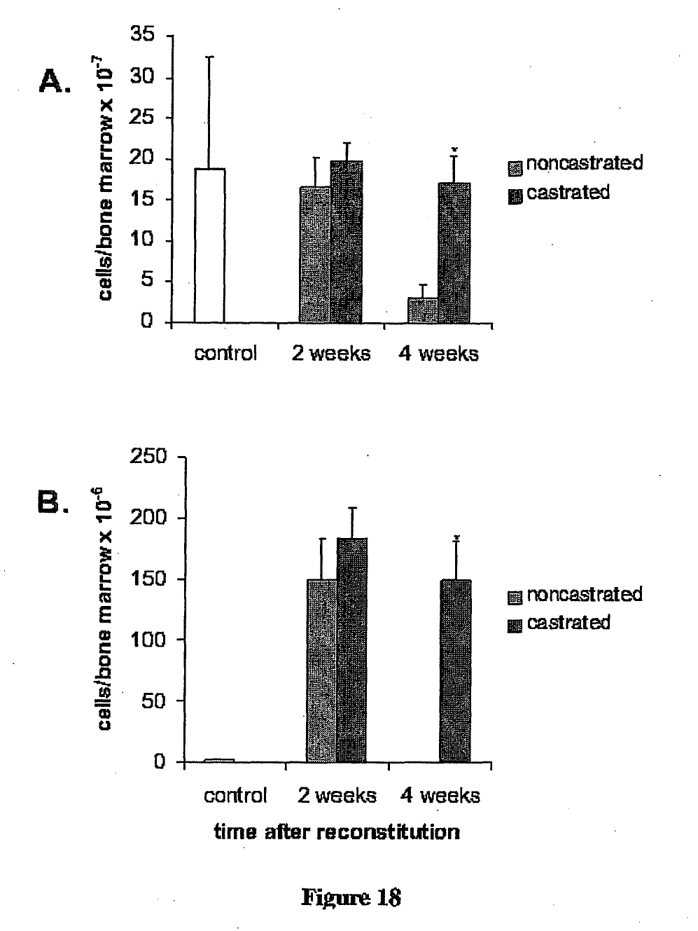

Cell numbers in the bone marrow of castrated and noncastrated reconstituted mice were compared to those of untreated age matched controls and are summarised in FIG. 18A. Bone marrow cell numbers were normal two and four weeks after reconstitution in castrated mice. Those of noncastrated mice were normal at two weeks but dramatically decreased at four weeks (p<0.05). Although, at this time point the noncastrated mice did not reconstitute with donor-derived cells.

Flow cytometeric analysis of the bone marrow with respect to CD45.2 (donor-derived antigen) established that no donor derived cells were detectable in the bone marrow of noncastrated mice 4 weeks after reconstitution, however, almost all the cells in the castrated mice were donor-derived at this time point (FIG. 18B).

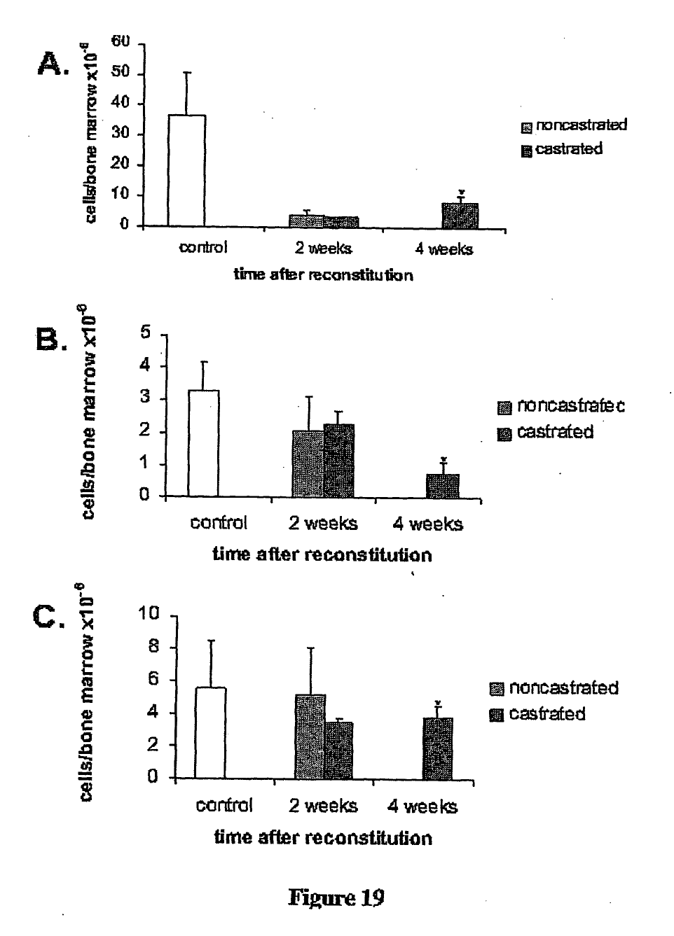

Two weeks after reconstitution the donor-derived T cell numbers of both castrated and noncastrated mice were markedly lower than those seen in the control mice (p<0.05). At 4 weeks there were no donor-derived T cells in the bone marrow of noncastrated mice and T cell number remained below control levels in castrated mice (FIG. 19A).

Donor-derived, myeloid and lymphoid dendritic cells were found at control levels in the bone marrow of noncastrated and castrated mice 2 weeks after reconstitution. Four weeks after treatment numbers decreased further in castrated mice and no donor-derived cells were seen in the noncastrated group (FIG. 19B).

Splenic Changes Following Lethal Irradiation, Foetal Liver Reconstitution and Castration

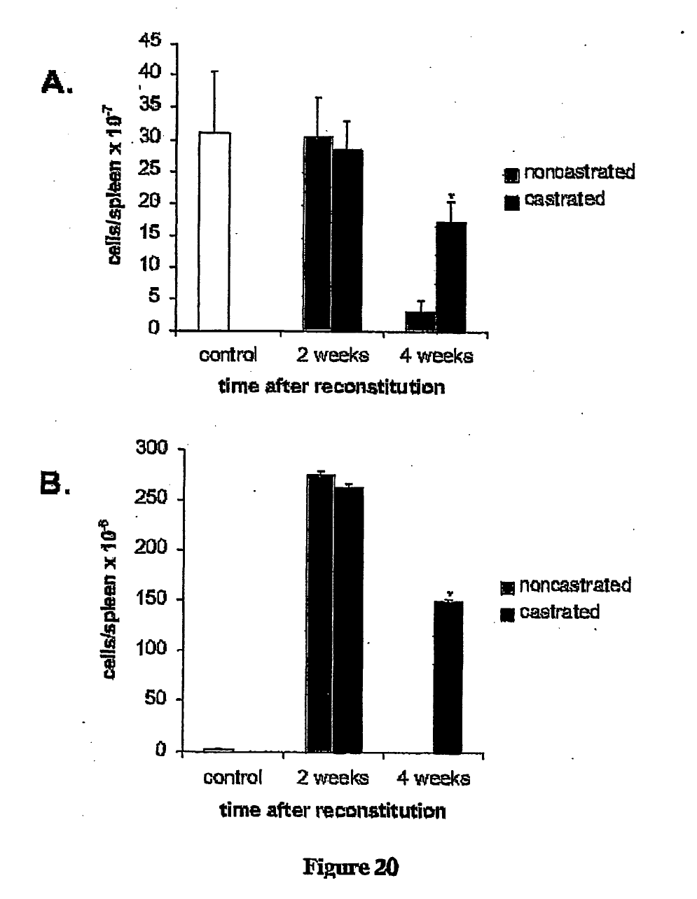

Spleen cell numbers of castrated and noncastrated reconstituted mice were compared to untreated age matched controls and the results are summarised in FIG. 20A. Two weeks after treatment, spleen cell numbers of both castrated and noncastrated mice were approximately 50% that of the control. By four weeks, numbers in castrated mice were approaching normal levels, however, those of noncastrated mice remained decreased. Analysis of CD45.2 (donor-derived) flow cytometry data demonstrated that there was no significant difference in the number of donor derived cells of castrated and noncastrated mice, 2 weeks after reconstitution (FIG. 20B). No donor derived cells were detectable in the spleens of noncastrated mice at 4 weeks, however, almost all the spleen cells in the castrated mice were donor derived.

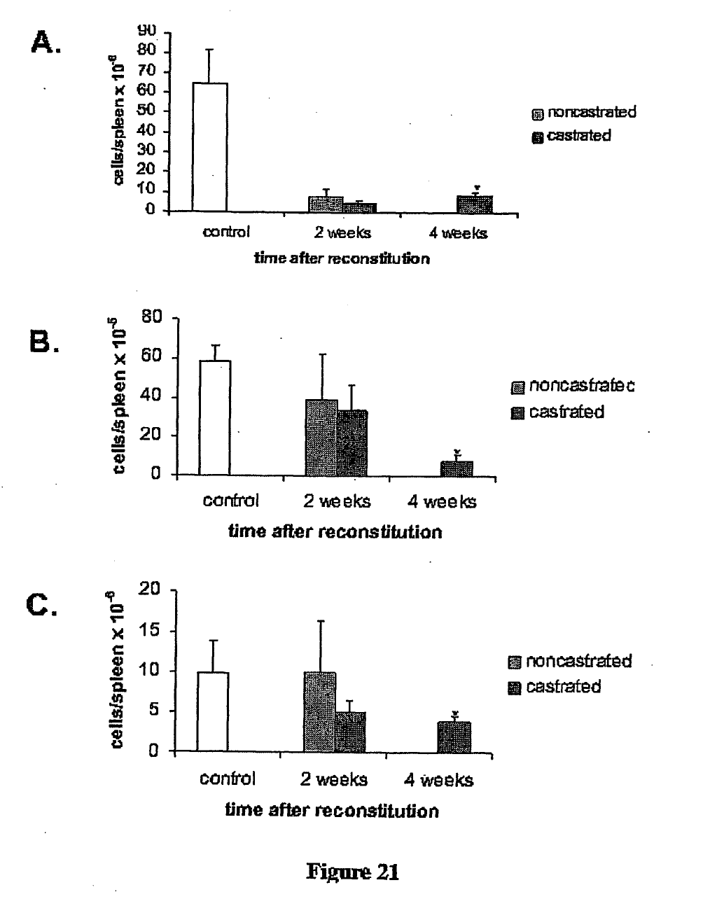

Two and four weeks after reconstitution there was a marked decrease in T cell number in both castrated and noncastrated mice (p<0.05) (FIG. 21A). Two weeks after foetal liver reconstitution donor-derived myeloid and lymphoid dendritic cells (FIGS. 21 A and B respectively) were found at control levels in noncastrated and castrated mice. At 4 weeks no donor derived dendritic cells were detectable in the spleens of noncastrated mice and numbers remained decreased in castrated mice.

The Effects of Lethal Irradiation, Foetal Liver Reconstitution and Castration on Mesenteric Lymph Node Numbers.

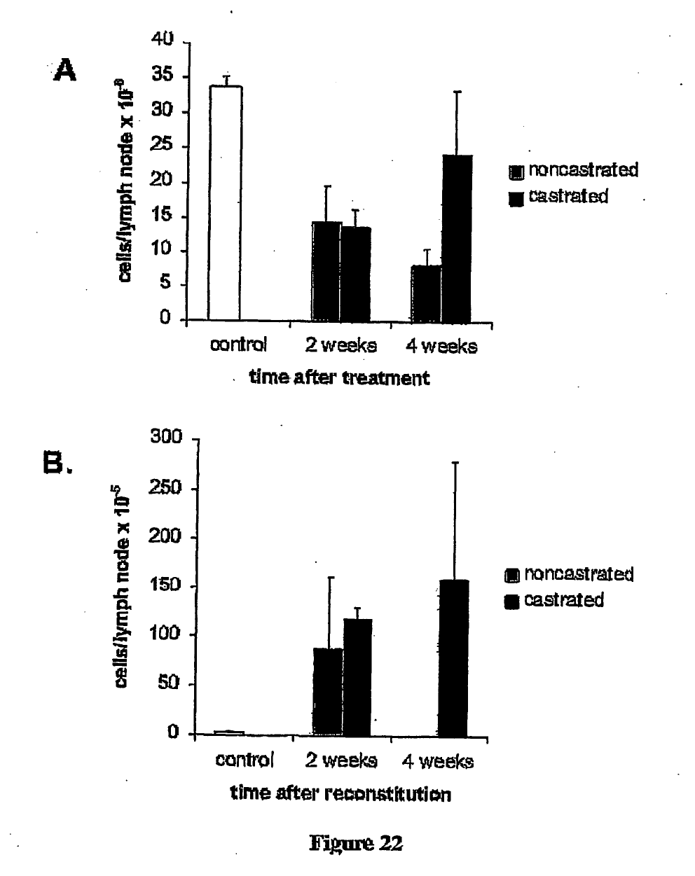

Lymph node cell numbers of castrated and noncastrated, reconstituted mice were compared to those of untreated age matched controls and are summarised in FIG. 22A. Two weeks after reconstitution cell numbers were at control levels in both castrated and noncastrated mice. Four weeks after reconstitution, cell numbers in castrated mice remained at control levels but those of noncastrated mice decreased significantly (FIG. 22B). Flow cytometry analysis with respect to CD45.2 suggested that there was no significant difference in the number of donor-derived cells, in castrated and noncastrated mice, 2 weeks after reconstitution (FIG. 22B). No donor derived cells were detectable in noncastrated mice 4 weeks after reconstitution. However, virtually all lymph node cells in the castrated mice were donor-derived at the same time point

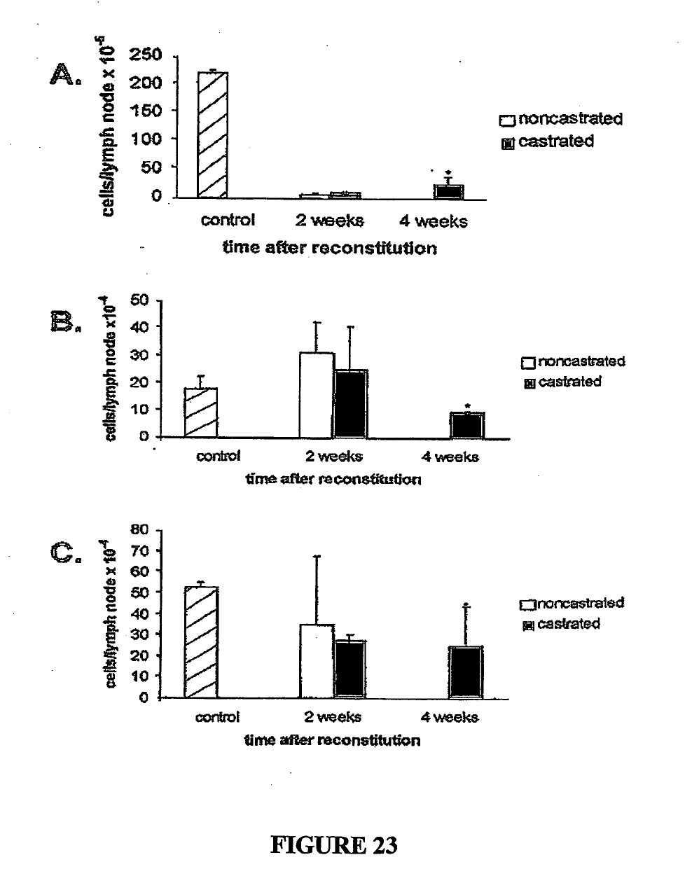

Two and four weeks after reconstitution donor-derived T cell numbers in both castrated and noncastrated mice were lower than control levels. At 4 weeks the numbers remained low in castrated mice and there were no donor-derived T cells in the lymph nodes of noncastrated mice (FIG. 23). Two weeks after foetal liver reconstitution donor-derived, myeloid and lymphoid dendritic cells were found at control levels in noncastrated and castrated mice (FIGS. 23 A & B respectively). Four weeks after treatment the number of donor-derived myeloid dendritic cells fell below the control, however, lymphoid dendritic cell number remained unchanged.

### GENERAL DISCUSSION OF THE EXAMPLES

We have shown that aged thymus, although severely atrophic, maintains its functional capacity with age, with T cell proliferation, differentiation and migration occurring at levels equivalent to the young adult mouse. Although thymic function is regulated by several complex interactions between the neuro-endocrine-immune axes, the atrophy induced by sex steroid production exerts the most significant and prolonged effects illustrated by the extent of thymus regeneration post-castration both of lymphoid and epithelial cell subsets.

Thymus weight is significantly reduced with age as shown previously (Hirokawa and Makinodan, 1975, Aspinall, 1997) and correlates with a significant decrease in thymocyte numbers. The stress induced by the castration technique, which may result in further thymus atrophy due to the actions of corticosteroids, is overridden by the removal of sex steroid influences with the 2-week castrate thymus increasing in cellularity by 20-30 fold from the pre-castrate thymus. By 3 weeks post-castration, the aged thymus shows a significant increase in both thymic size and cell number, surpassing that of the young adult thymus presumably due to the actions of sex steroids already exerting themselves in the 2 month old mouse.

Our data confirms previous findings that emphasise the continued ability of thymocytes to differentiate and maintain constant subset proportions with age (Aspinall, 1997). In addition, we have shown thymocyte differentiation to occur simultaneously post-castration indicative of a synchronous expansion in thymocyte subsets. Since thymocyte numbers are decreased significantly with age, proliferation of thymocytes was analysed to determine if this was a contributing factor in thymus atrophy.

Proliferation of thymocytes was not affected by age-induced thymic atrophy or by removal of sex-steroid influences post-castration with ˜14% of all thymocytes proliferating. However, the localisation of this division differed with age: the 2 month mouse thymus shows abundant division throughout the subcapsular and cortical areas (TN and DP T cells) with some division also occurring in the medulla. Due to thymic epithelial disorganisation with age, localisation of proliferation was difficult to distinguish but appeared to be less uniform in pattern than the young and relegated to the outer cortex. By 2 weeks post-castration, dividing thymocytes were detected throughout the cortex and were evident in the medulla with similar distribution to the 2 month thymus.

The phenotype of the proliferating population as determined by CD4 and CD8 analysis, was not altered with age or following castration. However, analysis of proliferation within thymocyte subpopulations, revealed a significant decrease in proliferation of both the TN and CD8⁺ cells with age. Further analysis within the TN subset on the basis of the markers CD44 and CD25, revealed a significant decrease in proliferation of the TN1 (CD44⁺CD25⁻) population which was compensated for by an increase in the TN2 (CD44⁻CD25⁺) population. These abnormalities within the TN population, reflect the findings by Aspinall (1997). Surprisingly, the TN subset was proliferating at normal levels by 2 weeks post-castration indicative of the immediate response of this population to the inhibition of sex-steroid action. Additionally, at both 2 weeks and 4 weeks post-castration, the proportion of CD8+ T cells that were proliferating was markedly increased from the control thymus, possibly indicating a role in the re-establishment of the peripheral T cell pool.

Thymocyte migration was shown to occur at a constant proportion of thymocytes with age conflicting with previous data by Scollay et al (1980) who showed a ten-fold reduction in the rate of thymocyte migration to the periphery. The difference in these results may be due to the difficulties in intrathymic FITC labelling of 2 year old thymuses or the effects of adipose deposition on FITC uptake. However, the absolute numbers of T cells migrating was decreased significantly as found by Scollay resulting in a significant reduction in ratio of RTEs to the peripheral T cell pool. This will result in changes in the periphery predominantly affecting the T cell repertoire (Mackall et al., 1995). Previous papers (Mackall et al, 1995) have shown a skewing of the T cell repertoire to a memory rather than naive T cell phenotype with age. The diminished T cell repertoire however, may not cope if the individual encounters new pathogens, possibly accounting for the rise in immunodeficiency in the aged. Obviously, there is a need to re-establish the T cell pool in immunocompromised individuals. Castration allows the thymus to repopulate the periphery through significantly increasing the production of naive T cells.

In the periphery, T cell numbers remained at a constant level as evidenced in the B:T cell ratios of spleen and lymph nodes, presumably due to peripheral homeostasis (Mackall et al., 1995; Berzins et al., 1998). However, disruption of cellular composition in the periphery was evident with the aged thymus showing a significant decrease in CD4:CD8 ratios from 2:1 in the young adult to 1:1 in the 2 year mouse, possibly indicative of the more susceptible nature of CD4⁺ T cells to age or an increase in production of CD8⁺ T cells from extrathymic sources. By 2 weeks post-castration, this ratio has been normalised, again reflecting the immediate response of the immune system to surgical castration.

The above findings have shown firstly that the aged thymus is capable of functioning in a nature equivalent to the pre-pubertal thymus. In this respect, T cell numbers are significantly decreased but the ability of thymocytes to differentiate is not disturbed. Their overall ability to proliferate and eventually migrate to the periphery is again not influenced by the age-associated atrophy of the thymus. However, two important findings were noted. Firstly, there appears to be an adverse affect on the TN cells in their ability to proliferate, correlating with findings by Aspinall (1997). This defect could be attributed to an inherent defect in the thymocytes themselves. Yet our data, and previous work has shown thymocyte differentiation, although diminished, still occurs and stem cell entry from the BM is also not affected with age (Hirokawa, 1998; Mackall and Gress, 1997). This implicates the thymic stroma as the target for sex steroid action and consequently abnormal regulation of this precursor subset of T cells. Secondly, the CD8⁺ T cells were significantly diminished in their proliferative capacity with age and, following castration, a significantly increased proportion of CD8⁺ T cells proliferated as compared to the 2 month mouse. The proliferation of mature T cells is thought to be a final step before migration (Suda and Zlotnik, 1992), such that a significant decrease in CD8⁺ proliferation would indicate a decrease in their migrational potential. This hypothesis is supported by our finding that the ratio of CD4:CD8 T cells in RTEs increased with age, indicative of a decrease in CD8 T cells migrating. Alternatively, if the thymic epithelium is providing the key factor for the CD8 T cell maintenance, whether a lymphostromal molecule or cytokine influence, this factor may be disturbed with increased sex-steroid production. By removing the influence of sex-steroids, the CD8 T cell population can again proliferate optimally. Thus, it was necessary to determine, in detail, the status of thymic epithelial cells pre- and post-castration.

The cortex appears to ‘collapse’ with age due to lack of thymocytes available to expand the network of epithelium. The most dramatic change in thymic epithelium post-castration was the increased network of cortical epithelium detected by MTS 44, illustrating the significant rise in thymocyte numbers. At 2 weeks post-castration, KNAs are abundant and appear to accommodate proliferating thymocytes indicating that thymocyte development is occurring at a rate higher than the epithelium can cope with. The increase in cortical epithelium appears to be due to stretching of the thymic architecture rather than proliferation of this subtype since no proliferation of the epithelium was noted with BrdU staining by immunofluorescence.

Medullary epithelium is not as susceptible to age influences most likely due to the lesser number of T cells accumulating in this area (>95% of thymocytes are lost at the DP stage due to selection events). However, the aged thymus shows severe epithelial cell disruption distinguished by a lack of distinction of the cortico-medullary junction with the medullary epithelium incorporating into the cortical epithelium. By 2 weeks post-castration, the medullary epithelium, as detected by MTS 10 staining is reorganised to some extent, however, subpockets are still present within the cortical epithelium. By 4 weeks post-castration, the cortical and medullary epithelium is completely reorganised with a distinct cortico-medullary junction similar to the young adult thymus.

Subtle changes were also observed following castration, most evident in the decreased expression of MHC class 11 and blood-thymus barrier antigens when compared to the pre-castrate thymus. MHCII (detected by MTS6) is increased in expression in the aged thymus possibly relating to a decrease in control by the developing thymocytes due to their diminished numbers. Alternatively, it may simply be due to lack of masking by the thymocytes, illustrated also in the post-irradiation thymus (Randle and Boyd, 1992) which is depleted of the DP thymocytes. Once thymocyte numbers are increased following castration, the antigen binding sites are again blocked by the accumulation of thymocytes thus decreasing detection by immunofluorescence. The antigens detecting the blood-thymus barrier (MTS12, 15 and 16) are again increased in the aged thymus and also revert to the expression in the young adult thymus post-castration. Lack of masking by thymocytes and the close proximity of the antigens due to thymic atrophy may explain this increase in expression. Alternatively, the developing thymocytes may provide the necessary control mechanisms over the expression of these antigens thus when these are depleted, expression is not controlled. The primordial epithelial antigens detected by MTS 20 and MTS 24 are increased in expression in the aged thymus but revert to subpockets of epithelium at the cortico-medullary junction post-castration. This indicates a lack of signals for this epithelial precursor subtype to differentiate in the aged mouse. Removing the block placed by the sex-steroids, these antigens can differentiate to express cortical epithelial antigens.

The above findings indicate a defect in the thymic epithelium rendering it incapable of providing the developing thymocytes with the necessary stimulus for, development. However, the symbiotic nature of the thymic epithelium and thymocytes makes it difficult to ascertain the exact pathway of destruction by the sex steroid influences. The medullary epithelium requires cortical T cells for its proper development and maintenance. Thus, if this population is diminished, the medullary thymocytes may not receive adequate signals for development This particularly seems to affect the CD8⁺ population. IRF^{-/-} mice show a decreased number of CD8⁺ T cells. It would therefore, be interesting to determine the proliferative capacity of these cells.

The defect in proliferation of the TN1 subset which was observed indicates that loss of cortical epithelium affects thymocyte development at the crucial stage of TCR gene rearrangement whereby the cortical epithelium provides factors such as IL-7 and SCF necessary for thymopoiesis (Godfrey and Zlotnik, 1990; Aspinall, 1997). Indeed, IL-7^{-/-}- and IL7R^{-/-} mice show similar thymic morphology to that seen in aged mice (Wiles et al., 1992; Zlotnik and Moore, 1995; von Freeden-Jeffry, 1995). Further work is necessary to determine the changes in IL-7 and IL-7R with age.

In conclusion, the aged thymus still maintains its functional capacity, however, the thymocytes that develop in the aged mouse are not under the stringent control by thymic epithelial cells as seen in the normal young mouse due to the lack of structural integrity of the thymic microenvironment Thus the proliferation, differentiation and migration of these cells will not be under optimal regulation and may result in the increased release of autoreactive/immunodysfunctional T cells in the periphery. The defects within both the TN and particularly, CD8⁺ populations, may result in the changes seen within the peripheral T cell pool with age. In addition, we have described in detail, the effects of castration on thymic epithelial cell development and reorganisation. The mechanisms underlying thymic atrophy utilising steroid receptor binding assays and the role of thymic epithelial subsets in thymus regeneration post-castration are currently under study. Restoration of thymus function by castration will provide an essential means for regenerating the peripheral T cell pool and thus in re-establishing immunity in immunosuppressed individuals.

The impact of castration on thymic structure and T cell production was investigated in animal models of immunodepletion. Specifically, Example 2 examined the effect of castration on the recovery of the immune system after sublethal irradiation and cyclophosphamide treatment These forms of immunodepletion act to inhibit DNA synthesis and therefore target rapidly dividing cells. In the thymus these cells are predominantly immature cortical thymocytes, however all subsets are effected (Fredrickson and Basch 1994). In normal healthy aged mice, the qualitative and quantitative deviations in peripheral T cells seldom lead to pathological states. However, major problems arise following severe depletion of T cells because of the reduced capacity of the thymus for T cell regeneration. Such insults occur in HIV/AIDS, and particularly following chemotherapy and radiotherapy in cancer treatment (Mackall et al. 1995).

In both sublethally irradiated and cyclophosphamide treated mice, castration markedly enhanced thymic regeneration. Castration was carried out on the same day as and seven days prior to immunodepletion in order to appraise the effect of the predominantly corticosteroid induced, stress response to surgical castration on thymic regeneration. Although increases in thymus cellularity and architecture were seen as early as one week after immunodepletion, the major differences were observed two weeks after castration. This was the case whether castration was performed on the same day or one week prior to immunodepletion.

Immunohistology demonstrated that in all instances, two weeks after castration the thymic architecture appeared phenotypically normal, while that of noncastrated mice was disorganised. Pan epithelial markers demonstrated that immunodepletion caused a collapse in cortical epithelium and a general disruption of thymic architecture in the thymii of noncastrated mice. Medullary markers supported this finding. Interestingly, one of the first features of castration-induced thymic regeneration was a marked upregulation in the extracellular matrix, identified by MTS 16.

Flow cytometry analysis data illustrated a significant increase in the number of cells in all thymocyte subsets in castrated mice, corresponding with the immunofluorescence. At each time point, there was a synchronous increase in all CD4, CD8 and αβ-TCR—defined subsets following immunodepletion and castration. This is an unusual but consistent result, since T cell development is a progressive process it was expected that there would be an initial increase in precursor cells (contained within the CD4⁻CD8⁻ gate) and this may have occurred before the first time point Moreover, since precursors represent a very small proportion of total thymocytes, a shift in their number may not have been detectable. The effects of castration on other cells, including macrophages and granulocytes were also analysed. In general there was little alteration in macrophage and granulocyte numbers within the thymus.

In both irradiation and cyclophosphamide models of immunodepletion thymocyte numbers peaked at every two weeks and decreased four weeks after treatment. Almost immediately after irradiation or chemotherapy, thymus weight and cellularity decreased dramatically and approximately 5 days later the first phase of thymic regeneration begun. The first wave of reconstitution (days 5-14) was brought about by the proliferation of radioresistant thymocytes (predominantly double negatives) which gave rise to all thymocyte subsets (Penit and Ezine 1989). The second decrease, observed between days 16 and 22 was due to the limited proliferative ability of the radioresistant cells coupled with a decreased production of thymic precursors by the bone marrow (also effected by irradiation). The second regenerative phase was due to the replenishment of the thymus with bone marrow derived precursors (Huiskamp et al. 0.1983).

It is important to note that in adult mice the development from a HSC is to a mature T cell takes approximately 28 days (Shortman et al. 1990).

Therefore, it is not surprising that little change was seen in peripheral T cells up to four weeks after treatment. The periphery would be supported by some thymic export, but the majority of the T cells found in the periphery up to four weeks after treatment would be expected to be proliferating cyclophosphamide or irradiation resistant clones expanding in the absence of depleted cells. Several long term changes in the periphery would be expected post-castration including, most importantly, a diversification of the TCR repertoire due to an increase in thymic export Castration did not effect the peripheral recovery of other leukocytes, including B cells, macrophages and granulocytes.

Example 4 shows the influence of castration on sygeneic and congenic bone marrow transplantation. Starzl et al. (1992) reported that microchimeras evident in lymphoid and nonlymphoid tissue were a good prognostic indicator for allograft transplantation. That is it was postulated that they were necessary for the induction of tolerance to the graft (Starzl et al. 1992). Donor-derived dendritic cells were present in these chimeras and were thought to play an integral role in the avoidance of graft rejection (Thomson and Lu 1999). Dendritic cells are known to be key players in the negative selection processes of thymus and if donor-derived dendritic cells were present in the recipient thymus, graft reactive T cells may be deleted.

In order to determine if castration would enable increased chimera formation, a study was performed using syngeneic foetal liver transplantation. The results showed an enhanced regeneration of thymii of castrated mice. These trends were again seen when the experiments were repeated using congenic (Ly5) mice. Due to the presence of congenic markers, it was possible to assess the chimeric status of the mice. As early as two weeks after foetal liver reconstitution there were donor-derived dendritic cells detectable in the thymus, the number in castrated mice being four-fold higher than that in noncastrated mice. Four weeks after reconstitution the noncastrated mice did not appear to be reconstituted with donor derived cells, suggesting that castration may in fact increase the probability of chimera formation. Given that castration not only increases thymic regeneration after lethal irradiation and foetal liver reconstitution and that it also increases the number of donor-derived dendritic cells in the thymus, along-side stem cell transplantation this approach increases the probability of graft acceptance.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

### REFERENCES

Aspinall, R. 1997. Age-associated thymic atrophy in the mouse is due to a deficiency affecting rearrangement of the TCR during intrathymic T cell development J. Immunol. 158:3037.
Berzins, S. P., Boyd, R. L. and Miller, J. F. A. P. 1998. The role of the thymus and recent thymic migrants in the maintenance of the adult peripheral lymphocyte pool. J Exp. Med. 187:1839.
Boyd, R. L., Tucek, C. L., Godfrey, D. I., Wilson, T. J, Davidson, N. J., Bean, A. G. D., Ladyman, H. M., Ritter, M. A. and Hugo, P. 1993. The thymic microenvironment. Immunology Today 14:445.
Bruijntjes, J. P., Kuper, C. J., Robinson, J. E. and Schutirman, H. J. 1993. Epithelium-free area in the thymic cortex of rats. Dev. Immunol. 3:113.
Carayon, P., and Bord, A. 1992. Identification of DNA-replicating lymphocyte subsets using a new method to label the bromo-deoxyuridine incorporated into the DNA. J. Imm. Methods 147:225.
Douek, D. C., McFarland, R. D., Keiser, P. H., Gage, E. A., Massey, J. M., Haynes, B. F., Polis, M. A., Haase, A. T., Feinberg, M. B., Sullivan, J. L., Jamieson, B. D., Zack, J. A., Picker, L. J. and Koup, R. A. 1998. Changes in thymic function with age and during the treatment of HIV infection. Nature 396:690.
Fredrickson, G. G. and Basch, R. S. 1994. Early thymic regeneration after irradiation. Development and Comparative Immunology 18:251.
George, A. J. and Ritter, M. A 1996. Thymic involution with ageing: obsolescence or good housekeeping? Immunol. Today 17:267.
Godfrey, D. I, Izon, D. J., Tucek C. L., Wilson, T. J. and Boyd, R. L. 1990. The phenotypic heterogeneity of mouse thymic stromal cells. Immunol. 70:66.
Godfrey, D. I, and Zlotnik, A. 1993. Control points in early T-cell development Immunol. Today 14:547.
Hirokawa, K. 1998. Immunity and Ageing. In Principles and Practice of Geriatric Medicine. M. Pathy, ed. John Wiley and Sons Ltd.
Hirokawa, K. and Makinodan, T. 1975. Thymic involution: the effect on T cell differentiation. J. Immunol 114:1659.
Hirokawa, K., Utsuyama M., Kasai, M., Kurashima, C., Ishijima, S. and Zeng, Y.-X. 1994. Understanding the mechanism of the age-change of thymic function to promote T cell differentiation. Immunology Letters 40:269.
Hobbs, M. V., Weigle, W. O., Noonan, D. J., Torbett, B. E., McEvilly, R. J., Koch, R. J., Cardenas, G. J. and Ernst, D. N. 1993. Patterns of cytokine gene expression by CD4+ T cells from young and old mice. J. Immunol. 150:3602.
Homo-Delarche, R. and Dardenne, M. 1991. The neuroendocrine-immune axis. Seminars in Immunopathology.
Huiskamp, R., Davids, J. A. G. and Vos, O. 1983. Short- and long-term effects of whole body irradiation with fission neutrons or x-rays on the thymus in CBA mice. Radiation Research 95:370.
Kendall, M. D. 1988. Anatomical and physiological factors influencing the thymic microenvironment. In Thymus Update I, Vol. 1. M. D. Kendall, and M. A. Ritter, eds. Harwood Academic Publishers, p. 27.
Kurashima, C, Utsuyama, M., Kasai, M., Ishijima, S. A., Konno, A. and Hirokawa, A. 1995. The role of thymus in the aging of Th cell subpopulations and age-associated alteration of cytokine production by these cells. Int. Immunol. 7:97.
Mackall, C. L. et. al. 1995. Age, thymopoiesis and CD4+ T-lymphocyte regeneration after intensive chemotherapy. New England J. Med. 332:143.
Mackall, C. L. and Gress, R. E. 1997. Thymic aging and T-cell regeneration. Immunol. Rev. 160:91.
Penit, C. and Ezine, S. 1989. Cell proliferation and thymocyte subset reconstitution in sublethally irradiated mice: compared kinetics of endogenous and intrathymically transferred progenitors. Proc. Natl. Acad. Sci, U.S.A. 86:5547.
Penit, C., Lucas, B., Vasseur, F., Rieker, T. and Boyd, R. L. 1996. Thymic medulla epithelial cells acquire specific markers by post-mitotic maturation. Dev. Immunol. 5:25.
Plosker, G. L. and Brogden, R. N. 1994. Leuprorelin. A review of its pharmacology and therapeutic use in prostatic cancer, endometriosis and other sex hormone-related disorders. Drugs 48:930.
Randle-Barrett, E. S. and Boyd, R. L. 1994. Thymic microenvironment and lymphoid responses to sublethal irradiation. Dev. Immunol. 4:1.
Scollay, R. G., Butcher, E. C. and Weissman, I. L. 1980. Thymus cell migration. Quantitative aspects of cellular traffic from the thymus to the periphery in mice. Eur. J. Immunol. 10:210.
Shortman, K., Egerton, M., Spangrude, G. J. and Scollay, R. 1990. The generation and fate of thymocytes. Seminars in Imumuno. 2:3.
Starzl, T. E., Demetris, A. J., Murase, N., Ricardi, C. and Truce, M. 1992. Cell migration, chimerism, and graft acceptance. Lancet 339:1579.
Suda, T., and Zlotnik, A. 1991. IL-7 maintains the T cell precursor potential of CD3⁻CD4⁻CD8⁻ thymocytes. J. Immunol. 146:3068.
Timm, J. A. and Thoman, M. L. 1999. Maturation of CD4+ lymphocytes in the aged microenviroment results in a memory-enriched population. J. Immunol. 162:711.
Thomson, A. W. and Lu, L. 1999. Are dendritic cells the key to liver transplant? Immunology Today 20:20.
Tosi, R., Kraft, R., Luzi, P., Cintorino, M., Fankhause, G., Hess, M. W. and Cottier, H. 1982. Involution pattern of the human thymus. 1. Size of the cortical area as a function of age. Clin. Exp. Immunol. 47:497.
van Ewijk, W., Rouse, R. V. and Weissman, I. L. 1980. Distribution of H-2 microenvironments in the mouse thymus. J. Histochem. Cytochem. 28:1089.
von Freeden-Jeffry, U., Vieira, P., Lucian, L. A., McNeil, T., Burdach, E. G. and Murray, R. 1995. Lymphopenia in interleukin (IL)-7 gene-deleted mice identifies IL-7 as a nonredundant cytokine. J. Exp. Med. 181:1519.
Wiles, M. V., Ruiz, P. and Imhof, B. A. 1992. Interleukin-7 expression during mouse thymus development Eur. J. Immunol 22:1037.
Zlotnik, A. and Moore, T. A. 1995. Cytokine production and requirements during T-cell development. Curr. Opin. Immunol. 7:206.

1. A method of treating a T-cell disorder in a post-pubertal human subject in need thereof, the method comprising disrupting sex steroid signalling in the human subject, and introducing into the subject allogeneic bone marrow or haemopoietic stem cells (HSC).

2. The method of claim 1, wherein the T cell disorder is selected from the group consisting of viral infections, a T cell proliferative disease, and any disease which causes a numerical or functional reduction in T cells.

3. The method of claim 2, wherein the viral infection is human immunodeficiency virus (HIV) infection.

4. The method of claim 3, wherein the subject has AIDS.

5. The method of claim 1, wherein the HSC are genetically modified prior to introduction into the human subject.

6. The method of claim 5, wherein the HSC are genetically modified such that the HSC and their progeny are resistant to infection and/or destruction with the HIV virus.

7. The method of claim 6, wherein the genetic modification comprises introducing into the HSC one or more nucleic acid molecules selected from the group consisting of a nucleic acid molecule which encodes an antiviral protein, an antisense construct, a ribozyme, a dsRNA and a catalytic nucleic acid molecule.

8. The method of claim 1, wherein the disruption of sex steroid signalling is achieved by physical or chemical castration.

9. The method of claim 1, wherein the disruption of sex steroid signalling is achieved by administration of a sex steroid analogue(s).

10. The method of claim 9, wherein the sex steroid analogue(s) is selected from the group consisting of the product flutamide sold under the trademark Eulexin® and dioxalan derivatives.

11. The method of claim 1, wherein disruption of sex steroid signalling is achieved by administration of a compound selected from the group consisting of leuprolide, goserelin, deslorelin, triptorelin, meterelin, buserelin, histrelin, nafarelin, lutrelin, and leuprorelin.

12. The method of claim 9, wherein the sex steroid analogue(s) is administered by a sustained peptide-release formulation.

13. The method of claim 1, wherein the HSC are enriched HSC.

14. The method of claim 1, wherein the HSC are autologous.

15. The method of claim 1, wherein the disruption of sex steroid signalling is achieved by administration of a luteinizing hormone-releasing hormone analogue(s).

16. The method of claim 1, wherein the HSC are non-autologous.

17. The method of claim 1, wherein sex steroid signalling to the thymus is disrupted.
